SEQUENCE LISTING (94 sequences)

SEQ ID NO: 1 - DNA, 33 bp, Homo sapiens
aactctctcccaagagcgaggccaactaaccaa

SEQ ID NO: 2 - DNA, 28 bp, Homo sapiens
cactcacgcaaacgggccaactaaccag

SEQ ID NO: 3 - DNA, 56 bp, Homo sapiens
acaactgggaagagccgtaagcgggaccgtcagaatcatgtaaaacgacggccagt

SEQ ID NO: 4 - DNA, 94 bp, Homo sapiens
  chromosome 3p24-p25; GenBank number af052155; Unigene description/ID SEC13 (S. cerevisiae)-like 1 (SEC13L1)/Hs.227949
cacccgccagctccaggactgccccttcctgggccaactaaccaaacaactgggaagagcccccaactccaacaggattattttcccaggagga

SEQ ID NO: 5 - DNA, 94 bp, Homo sapiens
  allele of SEQ ID NO 4; pivotal nucleotide (45)
cacccgccagctccaggactgccccttcctgggccaactaaccagacaactgggaagagcccccaactccaacaggattattttcccaggagga

SEQ ID NO: 6 - DNA, 33 bp, Homo sapiens
aactctctcccaagagcgattggcgagtgagtt

SEQ ID NO: 7 - DNA, 31 bp, Homo sapiens
cactcacgcaaacgggattggcgagtgagtg

SEQ ID NO: 8 - DNA, 55 bp, Homo sapiens
gagagccagctctgcacaagccatctcctgtccacgatgtaaaacgacggccagt

SEQ ID NO: 9 - DNA, 90 bp, Homo sapiens
  chromosome 4p16; GenBank number AC006230; Unigene description/ID RNA-binding protein S1, serine-rich domain (RNPSI)/Hs.75104
acacaccgcaccccaccactgtactctgaaattggcgagtgagttgagagccagctctgcggggtcatcacgcagccatggttgtgcctg

SEQ ID NO: 10 - DNA, 90 bp, Homo sapiens
  allele of SEQ ID NO 9; pivotal nucleotide (45)
acacaccgcaccccaccactgtactctgaaattggcgagtgagtggagagccagctctgcggggtcatcacgcagccatggttgtgcctg

SEQ ID NO: 11 - DNA, 33 bp, Homo sapiens
aactctctcccaagagcgattagcctgtggcaa

SEQ ID NO: 12 - DNA, 30 bp, Homo sapiens
cactcacgcaaacgggttagcctgtggcag

SEQ ID NO: 13 - DNA, 57 bp, Homo sapiens
taaagagaaactttgtgctccaagcgtggtccactccgatgtaaaacgacggccagt

SEQ ID NO: 14 - DNA, 88 bp, Homo sapiens
  chromosome 5q14-q21; GenBank number NM_000919; Unigene description/ID peptidylglycine alpha-amidating monooxygenase (PAM)/Hs.83920
ttctttggtgcctttcctgttcagcattcttagcctgtggcaataaagagaaactttgtgctacatgacgacaaagctgctaaatctc

SEQ ID NO: 15 - DNA, 88 bp, Homo sapiens
  allele of SEQ ID NO 14; pivotal nucleotide (43)
ttctttggtgcctttcctgttcagcattcttagcctgtggcagtaaagagaaactttgtgctacatgacgacaaagctgctaaatctc

SEQ ID NO: 16 - DNA, 34 bp, Homo sapiens
aactctctcccaagagcgatcgaggacagggact

SEQ ID NO: 17 - DNA, 31 bp, Homo sapiens
cactcacgcaaacgggtcgaggacagggacc

SEQ ID NO: 18 - DNA, 54 bp, Homo sapiens
ggcctgtctgtccactcaagcgattcctcgtgcgcatgtaaaacgacggccagt

SEQ ID NO: 19 - DNA, 98 bp, Homo sapiens
  chromosome 6p21.3; GenBank number AF029750; Unigene description/ID TAP binding protein (tapasin) (TAPBP)/Hs.179600
ccttaggtccctatgccggcgcggggttacagcagtggacagacaggccagtccctgtcctcgaggagcccatgatccgcggggagacaggcatttaa

SEQ ID NO: 20 - DNA, 98 bp, Homo sapiens
  allele of SEQ ID NO 19; pivotal nucleotide (50)
ccttaggtccctatgccggcgcggggttacagcagtggacagacaggccggtccctgtcctcgaggagcccatgatccgcggggagacaggcatttaa

SEQ ID NO: 21 - DNA, 36 bp, Homo sapiens
aactctctcccaagagcgattccttatttgattgct

SEQ ID NO: 22 - DNA, 33 bp, Homo sapiens
cactcacgcaaacgggttccttatttgattgcc

SEQ ID NO: 23 - DNA, 57 bp, Homo sapiens
gtatatggatacatggctgtcctgctgttgcatggcatctgtaaaacgacggccagt

SEQ ID NO: 24 - DNA, 98 bp, Homo sapiens
  chromosome Chr.6, 217.4 cR; GenBank number AW675467; Unigene description/ID splicing factor, arginine/serine-rich 3 (SFRS3)/Hs.167460
caagaaagtttacctttgctttaggtcataagttccttatttgattgctgtatatggatacatggctgttcgtgacattctttatgtgcaaatttgtg

SEQ ID NO: 25 - DNA, 98 bp, Homo sapiens
  allele of SEQ ID NO 24; pivotal nucleotide (49)
caagaaagtttacctttgctttaggtcataagttccttatttgattgccgtatatggatacatggctgttcgtgacattctttatgtgcaaatttgtg

SEQ ID NO: 26 - DNA, 31 bp, Homo sapiens
aactctctcccaagagcgatgacggctcacc

SEQ ID NO: 27 - DNA, 29 bp, Homo sapiens
cactcacgcaaacgggatgacggctcact

SEQ ID NO: 28 - DNA, 59 bp, Homo sapiens
gagagcatatctaaaaaacagatggcttttaggaacgcgcatgtaaaacgacggccagt

SEQ ID NO: 29 - DNA, 90 bp, Homo sapiens
  chromosome Chr.7, 22.53 cR; GenBank number AW665139; Unigene description/ID ESTs, weakly similar to myosin heavy chain  (containing ATP/GTP-binding site motif A (P-loop)  [Homo sapiens]/ Hs.73217
agttcaacaacatcttcttcttggattgacggatgacggctcaccgagagcatatctaaaaaacactctgcaaacatttggtcacatgca

SEQ ID NO: 30 - DNA, 90 bp, Homo sapiens
  allele of SEQ ID NO 29; pivotal nucleotide (45)
agttcaacaacatcttcttcttggattgacggatgacggctcactgagagcatatctaaaaaacactctgcaaacatttggtcacatgca

SEQ ID NO: 31 - DNA, 35 bp, Homo sapiens
aactctctcccaagagcgatacacggctaatcatt

SEQ ID NO: 32 - DNA, 32 bp, Homo sapiens
cactcacgcaaacgggtacacggctaatcatg

SEQ ID NO: 33 - DNA, 60 bp, Homo sapiens
gaaaattatgatctttgttaggatcaccgttaccgtcccgcatgtaaaacgacggccagt

SEQ ID NO: 34 - DNA, 97 bp, Homo sapiens
  chromosome 10q25-q26; GenBank number NM_006793; Unigene description/ID antioxidant protein 1 (AOP1)/Hs.75454
tttgtattaaactgaattttcttttaagctaacaaagatcataattttcaatgattagccgtgtaactcctgcaatgaatgtttatgtgattgaagc

SEQ ID NO: 35 - DNA, 97 bp, Homo sapiens
  allele of SEQ ID NO 34; pivotal nucleotide (50)
tttgtattaaactgaattttcttttaagctaacaaagatcataattttccatgattagccgtgtaactcctgcaatgaatgtttatgtgattgaagc

SEQ ID NO: 36 - DNA, 33 bp, Homo sapiens
aactctctcccaagagcgatccaaccaacttgg

SEQ ID NO: 37 - DNA, 31 bp, Homo sapiens
cactcacgcaaacgggatccaaccaacttgt

SEQ ID NO: 38 - DNA, 59 bp, Homo sapiens
ttctgcttcaataaatcttcgcaagacaggatttaggcgcatgtaaaacgacggccagt

SEQ ID NO: 39 - DNA, 70 bp, Homo sapiens
  chromosome 10p13; GenBank number M25246; Unigene description/ID vimentin (VIM)/Hs.2064
tgcttttttttttccagcaagtatccaaccaacttggttctgcttcaataaatctttggaaaaactcaaa

SEQ ID NO: 40 - DNA, 70 bp, Homo sapiens
  allele of SEQ ID NO 39; pivotal nucleotide (37)
tgcttttttttttccagcaagtatccaaccaacttgtttctgcttcaataaatctttggaaaaactcaaa

SEQ ID NO: 41 - DNA, 33 bp, Homo sapiens
aactctctcccaagagcgaccgctctgaccacc

SEQ ID NO: 42 - DNA, 30 bp, Homo sapiens
cactcacgcaaacgggccgctctgaccact

SEQ ID NO: 43 - DNA, 55 bp, Homo sapiens
gacaggcagagcaaaggtgctggctggattatggcgatgtaaaacgacggccagt

SEQ ID NO: 44 - DNA, 93 bp, Homo sapiens
  chromosome Chr.11, 540.2 cR, YAC cont; GenBank number AW663645; Unigene description/ID ESTs, weakly similar to Phospholemman precursor /Hs.3807
ggagcctgctgagtctccaacccacctcgctcaccgctctgaccaccgacaggcagagcaaaggatgcgggagttgcctctgctgcccatcta

SEQ ID NO: 45 - DNA, 93 bp, Homo sapiens
  allele of SEQ ID NO 44; pivotal nucleotide (47)
ggagcctgctgagtctccaacccacctcgctcaccgctctgaccactgacaggcagagcaaaggatgcgggagttgcctctgctgcccatcta

SEQ ID NO: 46 - DNA, 34 bp, Homo sapiens
aactctctcccaagagcgattaggtgctaaaccg

SEQ ID NO: 47 - DNA, 31 bp, Homo sapiens
cactcacgcaaacgggttaggtgctaaacca

SEQ ID NO: 48 - DNA, 56 bp, Homo sapiens
tttattttccacggatggaacgatcacgtgcgcaacgatgtaaaacgacggccagt

SEQ ID NO: 49 - DNA, 97 bp, Homo sapiens
  chromosome Chr.11, D11S1357-D11S9; GenBank number AW593045; Unigene description/ID ubiquitin-conjugating enzyme E2L 6/Hs.169895
tgagtcagccaagccactgatgggaatatacagatttaggtgctaaaccgtttattttccacggatgagtcacaatctgaagaatcaaacttccatc

SEQ ID NO: 50 - DNA, 97 bp, Homo sapiens
  allele of SEQ ID NO 49; pivotal nucleotide (50)
tgagtcagccaagccactgatgggaatatacagatttaggtgctaaaccatttattttccacggatgagtcacaatctgaagaatcaaacttccatc

SEQ ID NO: 51 - DNA, 31 bp, Homo sapiens
cactcacgcaaacgggttggcagcatcttcc

SEQ ID NO: 52 - DNA, 55 bp, Homo sapiens
ttgcctgtgataagttgcaagcacagcgatggctgattgtaaaacgacggccagt

SEQ ID NO: 53 - DNA, 55 bp, Homo sapiens; identical to SEQ ID NO: 52

SEQ ID NO: 54 - DNA, 94 bp, Homo sapiens
  chromosome Chr.12, 312.3 cR, Chr.16 D; GenBank number AW498942; Unigene description/ID tubulin, alpha, brain-specific/Hs.248323
tgccaatggtgtagtgccctcgggcatagttattggcagcatcttctttgcctgtgataagttgctcagggtggaagagctggcggtaggtgcc

SEQ ID NO: 55 - DNA, 94 bp, Homo sapiens
  allele of SEQ ID NO54; pivotal nucleotide (47)
tgccaatggtgtagtgccctcgggcatagttattggcagcatcttccttgcctgtgataagttgctcagggtggaagagctggcggtaggtgcc

SEQ ID NO: 56 - DNA, 34 bp, Homo sapiens
aactctctcccaagagcgatgtgcagggaatcat

SEQ ID NO: 57 - DNA, 32 bp, Homo sapiens
cactcacgcaaacgggatgtgcagggaatcac

SEQ ID NO: 58 - DNA, 58 bp, Homo sapiens
tttgctggattagaggacagttcgcaaggctggctggacatgtaaaacgacggccagt

SEQ ID NO: 59 - DNA, 97 bp, Homo sapiens
  chromosome hr. 17, D17S922-D17S79; GenBank number AW592223; Unigene description/ID ESTs, weakly similar to C. elegans sulphatases /Hs.12124
tagacccactgatcctgttactctgcttgtctctggtgtgcagggaatcattttgctggattagaggaaaggtgccgccgtctgtttccatgacttc

SEQ ID NO: 60 - DNA, 97 bp, Homo sapiens
  allele of SEQ ID NO 59; pivotal nucleotide (51)
tagacccactgatcctgttactctgcttgtctctggtgtgcagggaatcactttgctggattagaggaaaggtgccgccgtctgtttccatgacttc

SEQ ID NO: 61 - DNA, 36 bp, Homo sapiens
aactctctcccaagagcgattaaaagagcaaagttt

SEQ ID NO: 62 - DNA, 34 bp, Homo sapiens
cactcacgcaaacgggattaaaagagcaaagtta

SEQ ID NO: 63 - DNA, 56 bp, Homo sapiens
cccctccctttcttacagttcgcactcgcaactccgcatgtaaaacgacggccagt

SEQ ID NO: 64 - DNA, 99 bp, Homo sapiens
  chromosome Chr.18, 19.6 cR; GenBank number AW576208; Unigene description/ID  KIAA0249 gene product/ Hs. 166318
agatgaaaactactcttttggttttgtttgaaagtaagaaagggaggggaaactttgctcttttaataattatgttcagcctatgatgaagtatttgat

SEQ ID NO: 65 - DNA, 99 bp, Homo sapiens
  allele of SEQ ID NO 64; pivotal nucleotide (50)
agatgaaaactactcttttggttttgtttgaaagtaagaaagggaggggtaactttgctcttttaataattatgttcagcctatgatgaagtatttgat

SEQ ID NO: 66 - DNA, 19 bp, Homo sapiens
  primer
aactctctcccaagagcga

SEQ ID NO: 67 - DNA, 16 bp, Homo sapiens
  primer
cactcacgcaaacggg

SEQ ID NO: 68 - DNA, 18 bp, Homo sapiens
  primer
actggccgtcgttttaca

SEQ ID NO: 69 - DNA, 94 bp, Homo sapiens
  complement of SEQ ID NO 4
tcctcctgggaaaataatcctgttggagttgggggctcttcccagttgtttggttagttggcccaggaaggggcagtcctggagctggcgggtg

SEQ ID NO: 70 - DNA, 90 bp, Homo sapiens
  complement of SEQ ID NO 9
caggcacaaccatggctgcgtgatgaccccgcagagctggctctcaactcactcgccaatttcagagtacagtggtggggtgcggtgtgt

SEQ ID NO: 71 - DNA, 88 bp, Homo sapiens
  complement of SEQ ID NO 14
gagatttagcagctttgtcgtcatgtagcacaaagtttctctttattgccacaggctaagaatgctgaacaggaaaggcaccaaagaa

SEQ ID NO: 72 - DNA, 98 bp, Homo sapiens
  complement of SEQ ID NO 19
ttaaatgcctgtctccccgcggatcatgggctcctcgaggacagggactggcctgtctgtccactgctgtaaccccgcgccggcatagggacctaagg

SEQ ID NO: 73 - DNA, 99 bp, Homo sapiens
  complement of SEQ ID NO 24
cacaaatttgcacataaagaatgtcacgaacagccatgtatccatatacaggcaatcaaataaggaacttatgacctaaagcaaaggtaaactttcttg

SEQ ID NO: 74 - DNA, 90 bp, Homo sapiens
  complement of SEQ ID NO 29
tgcatgtgaccaaatgtttgcagagtgttttttagatatgctctcggtgagccgtcatccgtcaatccaagaagaagatgttgttgaact

SEQ ID NO: 75 - DNA, 97 bp, Homo sapiens
  complement of SEQ ID NO 34
gcttcaatcacataaacattcattgcaggagttacacggctaatcattgaaaattatgatctttgttagcttaaaagaaaattcagtttaatacaaa

SEQ ID NO: 76 - DNA, 70 bp, Homo sapiens
  complement of SEQ ID NO 39
tttgagtttttccaaagatttattgaagcagaaccaagttggttggatacttgctggaaaaaaaaaagca

SEQ ID NO: 77 - DNA, 93 bp, Homo sapiens
  complement of SEQ ID NO 44
tagatgggcagcagaggcaactcccgcatcctttgctctgcctgtcggtggtcagagcggtgagcgaggtgggttggagactcagcaggctcc

SEQ ID NO: 78 - DNA, 97 bp, Homo sapiens
  complement of SEQ ID NO 49
gatggaagtttgattcttcagattgtgactcatccgtggaaaataaacggtttagcacctaaatctgtatattcccatcagtggcttggctgactca

SEQ ID NO: 79 - DNA, 94 bp, Homo sapiens
  complement of SEQ ID NO 54
ggcacctaccgccagctcttccaccctgagcaacttatcacaggcaaagaagatgctgccaataactatgcccgagggcactacaccattggca

SEQ ID NO: 80 - DNA, 97 bp, Homo sapiens
  complement of SEQ ID NO 59
gaagtcatggaaacagacggcggcacctttcctctaatccagcaaaatgattccctgcacaccagagacaagcagagtaacaggatcagtgggtcta

SEQ ID NO: 81 - DNA, 99 bp, Homo sapiens
  complement of SEQ ID NO 64
atcaaatacttcatcataggctgaacataattattaaaagagcaaagtttcccctccctttcttactttcaaacaaaaccaaaagagtagttttcatct

SEQ ID NO: 82 - DNA, 94 bp, Homo sapiens
  complement of SEQ ID NO 5
tcctcctgggaaaataatcctgttggagttgggggctcttcccagttgtctggttagttggcccaggaaggggcagtcctggagctggcgggtg

SEQ ID NO: 83 - DNA, 90 bp, Homo sapiens
  complement of SEQ ID NO 10
caggcacaaccatggctgcgtgatgaccccgcagagctggctctccactcactcgccaatttcagagtacagtggtggggtgcggtgtgt

SEQ ID NO: 84 - DNA, 88 bp, Homo sapiens
  complement of SEQ ID NO 15
gagatttagcagctttgtcgtcatgtagcacaaagtttctctttactgccacaggctaagaatgctgaacaggaaaggcaccaaagaa

SEQ ID NO: 85 - DNA, 98 bp, Homo sapiens
  complement of SEQ ID NO 2O
ttaaatgcctgtctccccgcggatcatgggctcctcgaggacagggaccggcctgtctgtccactgctgtaaccccgcgccggcatagggacctaagg

SEQ ID NO: 86 - DNA, 98 bp, Homo sapiens
  complement of SEQ ID NO 25
cacaaatttgcacataaagaatgtcacgaacagccatgtatccatatacggcaatcaaataaggaacttatgacctaaagcaaaggtaaactttcttg

SEQ ID NO: 87 - DNA, 90 bp, Homo sapiens
  complement of SEQ ID NO 30
tgcatgtgaccaaatgtttgcagagtgttttttagatatgctctcagtgagccgtcatccgtcaatccaagaagaagatgttgttgaact

SEQ ID NO: 88 - DNA, 97 bp, Homo sapiens
  complement of SEQ ID NO 35
gcttcaatcacataaacattcattgcaggagttacacggctaatcatggaaaattatgatctttgttagcttaaaagaaaattcagtttaatacaaa

SEQ ID NO: 89 - DNA, 70 bp, Homo sapiens
  complement of SEQ ID NO 40
tttgagtttttccaaagatttattgaagcagaaacaagttggttggatacttgctggaaaaaaaaaagca

SEQ ID NO: 90 - DNA, 93 bp, Homo sapiens
  complement of SEQ ID NO 45
tagatgggcagcagaggcaactcccgcatcctttgctctgcctgtcagtggtcagagcggtgagcgaggtgggttggagactcagcaggctcc

SEQ ID NO: 91 - DNA, 97 bp, Homo sapiens
  complement of SEQ ID NO 50
gatggaagtttgattcttcagattgtgactcatccgtggaaaataaatggtttagcacctaaatctgtatattcccatcagtggcttggctgactca

SEQ ID NO: 92 - DNA, 94 bp, Homo sapiens
  complement of SEQ ID NO 55
ggcacctaccgccagctcttccaccctgagcaacttatcacaggcaaggaagatgctgccaataactatgcccgagggcactacaccattggca

SEQ ID NO: 93 - DNA, 97 bp, Homo sapiens
  complement of SEQ ID NO 60
gaagtcatggaaacagacggcggcacctttcctctaatccagcaaagtgattccctgcacaccagagacaagcagagtaacaggatcagtgggtcta

SEQ ID NO: 94 - DNA, 99 bp, Homo sapiens
  complement of SEQ ID NO 65
atcaaatacttcatcataggctgaacataattattaaaagagcaaagttacccctccctttcttactttcaaacaaaaccaaaagagtagttttcatct

(19) United States
(12) Patent Application Publication
(10) Pub. No.: US 2006/0263794 A1
(43) Pub. Date: Nov. 23, 2006

(54) METHODS FOR DETECTING TARGET NUCLEIC ACIDS USING COUPLED LIGATION AND AMPLIFICATION

(75) Inventor: Hans Michael Wenz, Redwood City, CA (US)

Correspondence Address: MILA KASAN, PATENT DEPT.;APPLIED BIOSYSTEMS, 850 LINCOLN CENTRE DRIVE, FOSTER CITY, CA, 94404, US

(73) Assignee: Applera Corporation, Foster City, CA (US)

(21) Appl. No.: 11/277,348

(22) Filed: Mar. 23, 2006

Related U.S. Application Data
(62) Division of application No. 09/724,755, filed on Nov. 28, 2000.
Application No. 09/724,755 is a division of application No. 09/584,905, filed on May 30, 2000.

(51) Int. Cl. C12Q 1/68; C12P 19/34

(52) U.S. Cl. 435/6; 435/91.2; 536/25.32

(57) ABSTRACT

The present invention relates to the detection of nucleic acid sequences using coupled ligation and amplification. The coupling of ligation and amplification allows multiplex detection of nucleic acid sequences. The invention also relates to methods, reagents, and kits that employ addressable-support specific sequences in detecting nucleic acid sequences.

### FIELD OF THE INVENTION

The present invention generally relates to the detection of nucleic acid sequences using coupled ligation and amplification reactions. The invention also relates to methods, reagents, and kits for detecting nucleic acid sequences.

### BACKGROUND OF THE INVENTION

The detection of nucleic acid sequences in a sample containing one or more sequences is a well-established technique in molecular biology. The entire sequence of the human genome will soon be known, allowing the identification and detection of numerous genetic diseases and for screening individuals for predisposition to genetic disease. Additionally, the detection of cancer and many infectious diseases, such as AIDS and hepatitis, routinely includes screening biological samples for the presence or absence of diagnostic nucleic acid sequences. Detecting nucleic acid sequences is also critical in forensic science, paternity testing, genetic counseling, and organ transplantation.

Frequently sequence detection is hampered due to low target copy number. Target sequences may be amplified using conventional techniques such as polymerase chain reaction (PCR), ligase detection reaction (LDR), and ligase chain reaction (LCR), followed by a standard detection procedure such as blotting or microarray detection. For example, microarrays have been used to detect LDR products that are created with probes containing array-specific sequences (Barany et al., PCT Publication No. WO 97/31256, published Aug. 28, 1997). Descriptions of these conventional amplification techniques can be found, among other places, in H. Ehrlich et al.,Science, 252:1643-50 (1991), M. Innis et al., PCR Protocols: A Guide to Methods and Applications, Academic Press, New York, N.Y. (1990), R. Favis et al., Nature Biotechnology 18:561-64 (2000), and H. F. Rabenau et al., Infection 28:97-102 (2000).

One variation of these basic amplification techniques is multiplex PCR, wherein multiple target sequences are simultaneously amplified using multiple sets of primers (see, e.g., H. Geada et al., Forensic Sci. Int. 108:31-37 (2000) and D. G. Wang et al., Science 280:1077-82 (1998)). Another variation involves combining LDR with PCR for detecting nucleic acid sequence differences (see, e.g., Msuih et al., J. Clin. Micro. 34:501-07,1996; U.S. Pat. No. 6,027,889).

Conventional nucleic acid detection methods, however, may be burdensome, time-consuming, or impractical, e.g., for high-throughput screening, especially when target sequences must first be amplified. There is a growing need for accurate, efficient and low cost methods, reagents, and kits for the simultaneous detection of multiple target sequences in a sample that is highly multiplexable. Fields where such needs apply include genetic testing, disease detection, and forensics. The inventions described herein may be used to detect one or more target sequences in a timely, reliable and cost-efficient manner.

### SUMMARY OF THE INVENTION

The present invention is directed to methods, reagents, and kits for detecting one or more nucleic acid sequences in a sample using coupled ligation and amplification reactions. Amplified ligation products, diagnostic for the presence or absence of target sequences in a sample, are hybridized to addressable supports that are designed to detect specific nucleic acid sequences. Alternatively, amplified ligation products, diagnostic for the presence or absence of target sequences in a sample, comprising a specific length or molecular weight are separated based on molecular weight or length or mobility to detect specific nucleic acid sequences.

In certain embodiments, the sample preferably comprises genomic DNA. Within the scope of the invention is large-scale multiplex analysis of polynucleotide or oligonucleotide sequences (target sequences) in a sample comprising, for example, but not limited to, multiple polymorphic loci.

In certain embodiments, the invention provides a method for detecting at least one target sequence in a sample comprising combining the sample with a probe set for each target sequence to be detected and optionally, a ligation agent to form a ligation reaction mixture. The probe set comprises (a) at least one first probe, comprising a target-specific portion and a 5′ primer-specific portion, and (b) at least one second probe, comprising a target-specific portion and a 3′ primer-specific portion. The probes in each set are suitable for ligation together when hybridized adjacent to one another on a complementary target sequence. Further, at least one probe in each probe set further comprises an addressable support-specific portion located between the primer-specific portion and the target-specific portion. This ligation reaction mixture is subjected to at least one cycle of ligation, wherein adjacently hybridizing complementary probes, under appropriate conditions, are ligated to one another to form a ligation product. The ligation product thus comprises the 5′ primer-specific portion, the target-specific portions, at least one addressable support-specific portion, and the 3′ primer-specific portion.

The ligation reaction mixture is combined with at least one primer set and a polymerase to form a first amplification reaction mixture. The primer set comprises (i) at least one first primer, comprising the sequence of the 5′ primer-specific portion of the ligation product, and (ii) at least one second primer, comprising a sequence complementary to the 3′ primer-specific portion of the ligation product. At least one primer of the primer set further comprises a reporter group. The first amplification reaction mixture is subjected to at least one cycle of amplification to generate a first amplification product comprising at least one reporter group. The addressable support-specific portions of the first amplification product are hybridized, under appropriate conditions, to support-bound capture oligonucleotides. The reporter group of the hybridized product is detected, indicating the presence of the target sequence in the sample.

In other embodiments, a method is provided for detecting at least one target sequence in a sample comprising combining the sample with a probe set for each target sequence and optionally, a ligation agent to form a ligation reaction mixture. The probe set comprises (a) at least one first probe, comprising a target-specific portion, and (b) at least one second probe, comprising a target-specific portion and a 3′ primer-specific portion. At least one probe in each probe set further comprises an addressable support-specific portion. The probes in each set are suitable for ligation together when hybridized adjacent to one another on a complementary target sequence.

The ligation reaction mixture is subjected to at least one cycle of ligation, wherein adjacently hybridizing complementary probes are ligated to one another to form a ligation product comprising the target-specific portions, at least one addressable support-specific portion, and the primer-specific portion. This ligation reaction mixture is combined with at least one primer comprising a sequence complementary to the primer-specific portion of the ligation product and a reporter group, and a polymerase, to form an extension reaction mixture.

A first amplification product, comprising at least one reporter group, is generated by subjecting the extension reaction mixture to at least one cycle of primer extension. The addressable support-specific portions of the first amplification product are hybridized to support-bound capture oligonucleotides. Detection of the reporter group indicates the presence of the corresponding target sequence in the sample.

In other embodiments, the first or the second probe of a probe set further comprise an addressable support-specific portion designed to allow hybridization with capture oligonucleotides on a support or to provide a unique molecular weight or length, or mobility, for example, but without limitation, electrophoretic mobility.

In yet other embodiments, ligation is performed non-enzymatically. While not limiting, non-enzymatic ligation includes chemical ligation, such as, autoligation and ligation in the presence of an “activating” and/or a reducing agent. Non-enzymatic ligation may utilize specific reactive groups on the respective 3′ and 5′ ends of the probes to be ligated.

In certain embodiments, single-stranded amplification products, suitable for hybridization with an addressable support, can be generated by several alternate methods including, without limitation, asymmetric PCR, asymmetric reamplification, nuclease digestion, and chemical denaturation. Detailed descriptions of such processes can be found, among other places, in Current Protocols in Molecular Biology, John Wiley & Sons, Inc. (1995 and supplements), Novagen Strandase™ Kit insert, Sambrook et al., Molecular Cloning, A Laboratory Manual, Cold Spring Harbor Press (1989), and Little et al., J. Biol. Chem. 242:672 (1967).

In certain embodiments of the invention, methods are provided to generate single-stranded sequences from amplification products using exonuclease digestion. The amplification product, comprising at least one 5′ terminal phosphate, is combined with an exonuclease to form a digestion reaction mixture. The digestion reaction mixture is incubated under conditions that allow the exonuclease to digest one strand of the amplification product, generating single stranded addressable support-specific portions.

In other embodiments of the invention, methods are provided to generate single-stranded sequences from amplification products by incorporating steps for asymmetric re-amplification. The first amplification product is combined with either at least one first primer or at least one second primer from each primer set, but not both, to generate a second amplification reaction mixture.

The skilled artisan will appreciate that in these asymmetric re-amplification methods the reporter group is a component of the primers in the second amplification reaction mixture, rather than the first amplification reaction mixture. The skilled artisan will also appreciate that additional polymerase may also be a component of the second amplification reaction mixture. Alternatively, residual polymerase from the first amplification mixture may be sufficient to synthesize the second amplification product.

The second amplification reaction mixture is then subjected to at least one cycle of amplification. Typically, only single-stranded amplicons are generated since the second amplification reaction mixture comprises only first or second primers from each primer set. The single-stranded second amplification product comprising a reporter group is hybridized with support-bound capture oligonucleotides. Detection of the reporter group indicates the presence of the corresponding target sequence in the sample.

Also within the scope by the inventive methods is the use of primer extension to generate single-stranded sequences that may be hybridized with the support-bound capture oligonucleotides or separated by molecular weight or length or mobility. According to these methods, the first amplification reaction mixture comprises at least one second primer, but no first primers from a primer set. Thus, only a single amplification product, the complement of the ligation product, is generated. This amplification product, comprising the complement of the addressable support-specific portion of the ligation product, is hybridized directly with the support-bound capture oligonucleotides. Alternatively, this amplification product is separated by molecular weight or length or mobility.

The person of ordinary skill will understand that single-stranded amplification product may also be generated using asymmetric PCR, wherein both the first and second primers for each primer set are provided, with one primer in excess relative to the other. Thus, unlike the primer extension process described above, either strand of a double-stranded ligation product can be amplified to generate single-stranded product, depending on which primer is supplied in excess.

In other embodiments, probes suitable for ligation are provided comprising: a 5′-end, a 3′ end, a target-specific portion, a primer-specific portion, and an addressable support-specific portion located between the primer-specific portion and the target-specific portion. In certain embodiments, probes suitable for ligation are provided that further comprise appropriate reactive groups for non-enzymatic ligation.

Kits for detecting at least one target sequence in a sample are also within the scope of the invention. In certain embodiments, the invention provides kits for detecting at least one target sequence in a sample comprising at least one probe set for each target sequence to be detected and optionally, a ligation agent. Each probe set comprises (a) at least one first probe, comprising a target-specific portion and a 5′ primer-specific portion, and (b) at least one second probe, comprising a target-specific portion and a 3′ primer-specific portion. The first and second probes in each set are suitable for ligation together when hybridized adjacent to one another on a complementary target sequence. At least one probe in each probe set further comprises an addressable support-specific portion located between the primer-specific portion and the target-specific portion.

In other embodiments, kits are provided that further comprise a set of nucleotide primers and a polymerase. The primer set comprises (i) at least one primer complementary to the 3′ primer-specific portion of the probe and optionally, (ii) at least one primer comprising the sequence of the 5′ primer-specific portion of the probe. At least one primer of the primer set further comprises a reporter group.

In other embodiments of the methods and kits, the polymerase is a thermostable polymerase, including, but not limited to, Taq, Pfu, Vent, Deep Vent, Pwo, UITma, and Tth polymerase and enzymatically active mutants and variants thereof. Descriptions of these polymerases may be found, among other places, at http://www.the-scientist.library.upenn.edu/yr1998/jan/profile 1_{—}980105.html

In certain embodiments the ligation agent is a ligase, including, without limitation, bacteriophage T4 or E. coli ligase. In other embodiments the ligase is a thermostable ligase, including, but not limited to Taq, Pfu, and Tth ligase. The skilled artisan will understand that any of a number of other polymerases and ligases could be used, including those isolated from thermostable or hyperthermostable prokaryotic, eucaryotic, or archael organisms. In yet other embodiments of the methods and kits of the invention, the ligation agent is an “activating” or reducing agent.

### BRIEF DESCRIPTION OF THE DRAWINGS

FIG. 1. Schematic showing a probe set according to certain embodiments of the invention.

Each probe includes a portion that is complementary or substantially complementary to the target (the “target-specific portion,” T-SP) and a portion that is complementary to or has the same sequence as a primer (the “primer-portion,” P-SP). At least one probe in each probe set further comprises an addressable support-specific portion (AS-SP) that is located between the target-specific portion and the primer-specific portion (here, probe Z).

Each probe set comprises at least one first probe and at least one second probe that are designed to hybridize with the target with the 3′ end of the first probe (here, probe A) immediately adjacent to and opposing the 5′ end of the second probe (here, probe Z).

FIG. 2 depicts a method for differentiating between two potential alleles in a target locus using certain embodiments of the invention.

FIG. 2(a) shows: (i) a target-specific probe set comprising two first probes, A and B, that differ in their primer-specific portions and their pivotal complement (T on the A probe and C on the B probe), and one second probe, Z, comprising an addressable support-specific portion and a primer-specific portion, and (ii) a target sequence, comprising pivotal nucleotide A.

FIG. 2(b) shows the three probes annealed to the target. The target-specific portion of probe A is fully complementary with the 3′ target region including the pivotal nucleotide. The pivotal complement of probe B is not complementary with the 3′ target region. The target-specific portion of probe B, therefore, contains a base-pair mismatch at the 3′ end. The target-specific portion of probe Z is fully complementary to the 5′ target region.

FIG. 2(c) shows ligation of probes A and Z to form ligation product A-Z. Probes B and Z are not ligated together to form a ligation product due to the mismatched pivotal complement on probe B.

FIG. 2(d) shows denaturing the double-stranded molecules to release the A-Z ligation product and unligated probes B and Z. FIG. 3. Schematic depicting certain embodiments of the inventive methods.

FIG. 3(a) depicts a target sequence and a probe set comprising two first probes, A and B, that differ in their primer-specific portions and their pivotal complements (here, T at the 3′ end probe A and G at the 3′end probe B), and one second probe, Z comprising the addressable support-specific portion (shown in wavy lines -vvvvv- upstream from primer-specific portion Z).

FIG. 3(b) depicts the A and Z probes hybridized to the target sequence under annealing conditions.

FIG. 3(c) depicts the ligation of the first and second probes in the presence of a ligation agent to form ligation product A-Z.

FIG. 3(d) depicts denaturing the ligation product:target complex to release a single-stranded ligation product; adding a primer set (PA*, PB*, and PZ), where the PA and PB primers comprise a reporter group (*); and annealing primer PZ to the ligation product.

FIG. 3(e) depicts the formation of a double-stranded nucleic acid product by extending the PZ primer in a template-dependent manner with a polymerase.

FIG. 3(f) depicts denaturing the double-stranded nucleic acid product to release two single-stranded molecules.

FIG. 3(g) shows the PA* and PZ primers annealed to their respective single-stranded molecules.

FIG. 3(h) shows both double-stranded amplification products.

FIG. 3(i) depicts both amplification products being denatured to release four single-stranded molecules including a single-stranded molecule comprising a reporter group, PA*.

FIG. 3(j) shows annealing the addressable support-specific portion of the single-stranded PA* amplification product to position 1 of the support.

FIG. 3(k) represents detecting the reporter group hybridized to position 1 of the support.

FIG. 4 depicts two or more ligation products comprising the same primer-specific portions and their respective primer sets.

FIG. 4(a) shows six ligation products and their respective primers. Each of the ligation products comprise a unique addressable support-specific portion (AS-SP). Two of the six ligation products comprise the same 5′ primer-specific portion and the same 3′ primer-specific portion, A and Z respectively. Consequently, only five primer sets (PA and PZ; PC and PX; PD and PW; PE and PV; and PF and PU) are required to amplify the six ligation products.

FIG. 4(b) shows six ligation products and their respective primers. Here most of the ligation products (4 of 6) comprise the same 5′ primer-specific portion and the same 3′ primer-specific portion, A and Z respectively. Consequently, only three primer sets (PA and PZ; PE and PV; and PF and PU) are required to amplify the six ligation products.

FIG. 4(c) shows six ligation products and their respective primers. Each of the six ligation products comprise unique addressable support-specific portions. All six ligation products comprise the same 5′ primer-specific portion and the same 3′ primer-specific portion, A and Z respectively. Consequently, only one primer set (PA and PZ) is required to amplify all six ligation products.

### DETAILED DESCRIPTION OF THE PREFERRED EMBODIMENTS

The section headings used herein are for organizational purposes only and are not to be construed as limiting the subject matter described. All references cited in this application are expressly incorporated by reference for any purpose to the same extent as if each reference was specifically and individually incorporated by reference. Likewise, the Sequence Listing, as originally filed with the specification, is incorporated by reference.

Definitions

The term “nucleoside” refers to a compound comprising a purine, deazapurine, or pyrimidine nucleobase, e.g., adenine, guanine, cytosine, uracil, thymine, 7-deazaadenine, 7-deazaguanosine, and the like, that is linked to a pentose at the 1′-position. When the nucleoside base is purine or 7-deazapurine, the pentose is attached to the nucleobase at the 9-position of the purine or deazapurine, and when the nucleobase is pyrimidine, the pentose is attached to the nucleobase at the 1-position of the pyrimidine, (e.g., Kornberg and Baker, DNA Replication, 2nd Ed. (Freeman, San Francisco, 1992)). The term “nucleotide” as used herein refers to a phosphate ester of a nucleoside, e.g., a triphosphate ester, wherein the most common site of esterification is the hydroxyl group attached to the C-5 position of the pentose. The term “nucleoside” as used herein refers to a set of compounds including both nucleosides and nucleotides.

The term “polynucleotide” means polymers of nucleotide monomers, including analogs of such polymers, including double and single stranded deoxyribonucleotides, ribonucleotides, -anomeric forms thereof, and the like. Monomers are linked by “internucleotide linkages,” e.g., phosphodiester linkages, where as used herein, the term “phosphodiester linkage” refers to phosphodiester bonds or bonds including phosphate analogs thereof, including associated counterions, e.g., H⁺, NH₄⁺, Na⁺, if such counterions are present. Whenever a polynucleotide is represented by a sequence of letters, such as “ATGCCTG,” it will be understood that the nucleotides are in 5′ to 3′ order from left to right and that “A” denotes deoxyadenosine, “C” denotes deoxycytidine, “G” denotes deoxyguanosine, and “T” denotes deoxythymidine, unless otherwise noted. Descriptions of how to synthesize oligonucleotides can be found, among other places, in U.S. Pat. Nos. 4,373,071; 4,401,796; 4,415,732; 4,458,066; 4,500,707; 4,668,777; 4,973,679; 5,047,524; 5,132,418; 5,153,319; and 5,262,530. Oligonucleotides can be of any length, but may preferably be 12 to 40 nucleotides in length, more preferably 15 to 35 nucleotides in length, and most preferably 17 to 25 nucleotides in length.

“Analogs” in reference to nucleosides and/or polynucleotides comprise synthetic analogs having modified nucleobase portions, modified pentose portions and/or modified phosphate portions, and, in the case of polynucleotides, modified internucleotide linkages, as described generally elsewhere (e.g., Scheit, Nucleotide Analogs (John Wiley, New York, (1980); Englisch, Angew. Chem. Int. Ed. Engl. 30:613-29 (1991); Agrawal, Protocols for Polynucleotides and Analogs, Humana Press (1994)). Generally, modified phosphate portions comprise analogs of phosphate wherein the phosphorous atom is in the +5 oxidation state and one or more of the oxygen atoms is replaced with a non-oxygen moiety, e.g., sulfur. Exemplary phosphate analogs include but are not limited to phosphorothioate, phosphorodithioate, phosphoroselenoate, phosphorod iselenoate, phosphoroanilothioate, phosphoranilidate, phosphoramidate, boronophosphates, including associated counterions, e.g., H⁺, NH₄⁺, Na⁺, if such counterions are present. Exemplary modified nucleobase portions include but are not limited to 2,6-diaminopurine, hypoxanthine, pseudouridine, C-5-propyne, isocytosine, isoguanine, 2-thiopyrimidine, and other like analogs. Particularly preferred nucleobase analogs are iso-C and iso-G nucleobase analogs available from Sulfonics, Inc., Alachua, Fla. (e.g., Benner, et al., U.S. Pat. No. 5,432,272) or LNA analogs (e.g., Koshkin et al., Tetrahedron 54:3607-30 (1998)). Exemplary modified pentose portions include but are not limited to 2′- or 3′-modifications where the 2′- or 3′-position is hydrogen, hydroxy, alkoxy, e.g., methoxy, ethoxy, allyloxy, isopropoxy, butoxy, isobutoxy and phenoxy, azido, amino or alkylamino, fluoro, chloro, bromo and the like. Modified internucleotide linkages include phosphate analogs, analogs having achiral and uncharged intersubunit linkages (e.g., Sterchak, E. P., et al., Organic Chem, 52:4202 (1987)), and uncharged morpholino-based polymers having achiral intersubunit linkages (e.g., U.S. Pat. No. 5,034,506). Preferred internucleotide linkage analogs include peptide nucleic acid (PNA), morpholidate, acetal, and polyamide-linked heterocycles. A particularly preferred class of polynucleotide analogs where a conventional sugar and internucleotide linkage has been replaced with a 2-aminoethylglycine amide backbone polymer is PNA (e.g., Nielsen et al., Science, 254:1497-1500 (1991); Egholm et al., J. Am. Chem. Soc., 114:1895-1897 (1992)).

The term “reporter group” as used herein refers to any tag, label, or identifiable moiety. The skilled artisan will appreciate that many reporter groups may be used in the present invention. For example, reporter groups include, but are not limited to, fluorophores, radioisotopes, chromogens, enzymes, antigens, heavy metals, dyes, magnetic probes, phosphorescence groups, chemiluminescent groups, and electrochemical detection moieties. Reporter groups also include elements of multi-element indirect reporter systems, e.g., biotin/avidin, antibody/antigen, ligand/receptor, enzyme/substrate, and the like, in which the element interacts with other elements of the system in order to effect a detectable signal. One exemplary multi-element reporter system includes a biotin reporter group attached to a primer and an avidin conjugated with a fluorescent label. Detailed protocols for methods of attaching reporter groups to oligonucleotides and polynucleotides can be found in, among other places, G. T. Hermanson, Bioconjugate Techniques, Academic Press, San Diego, Calif. (1996) and S. L. Beaucage et al., Current Protocols in Nucleic Acid Chemistry, John Wiley & Sons, New York, N.Y. (2000).

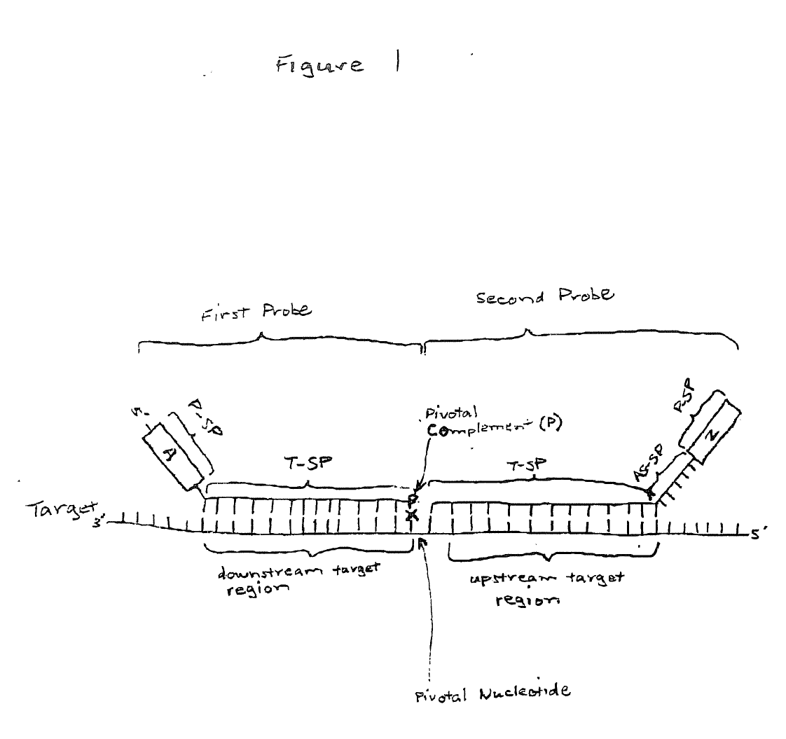

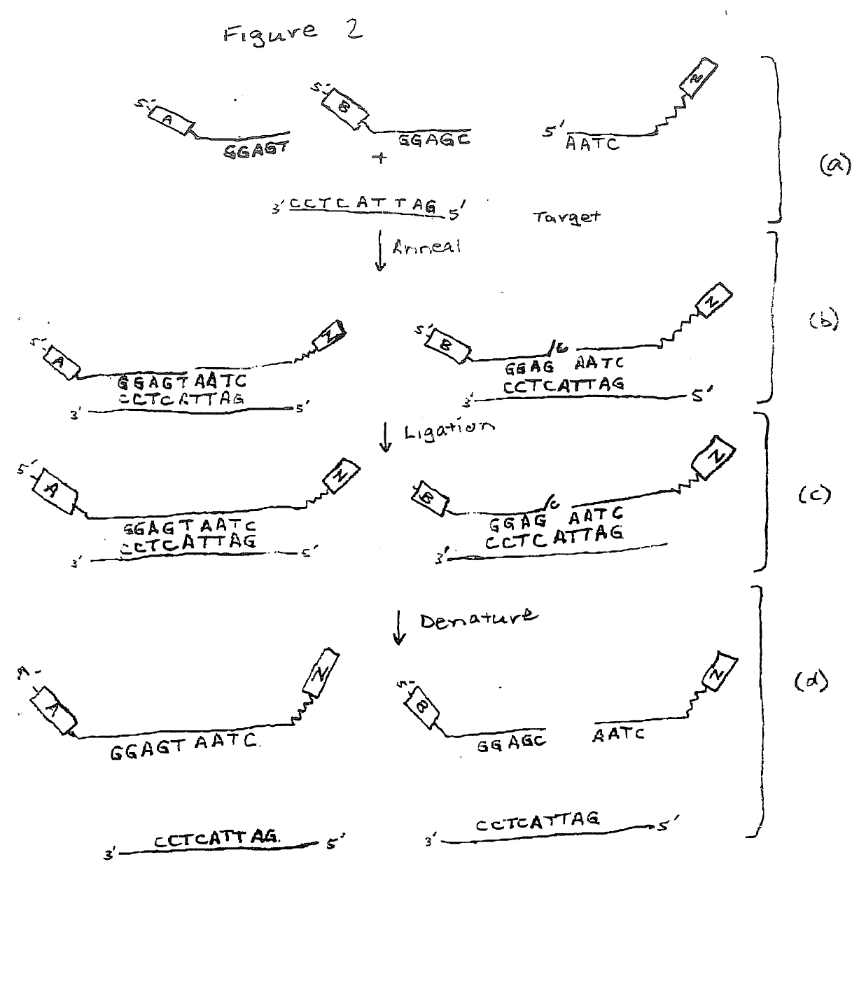

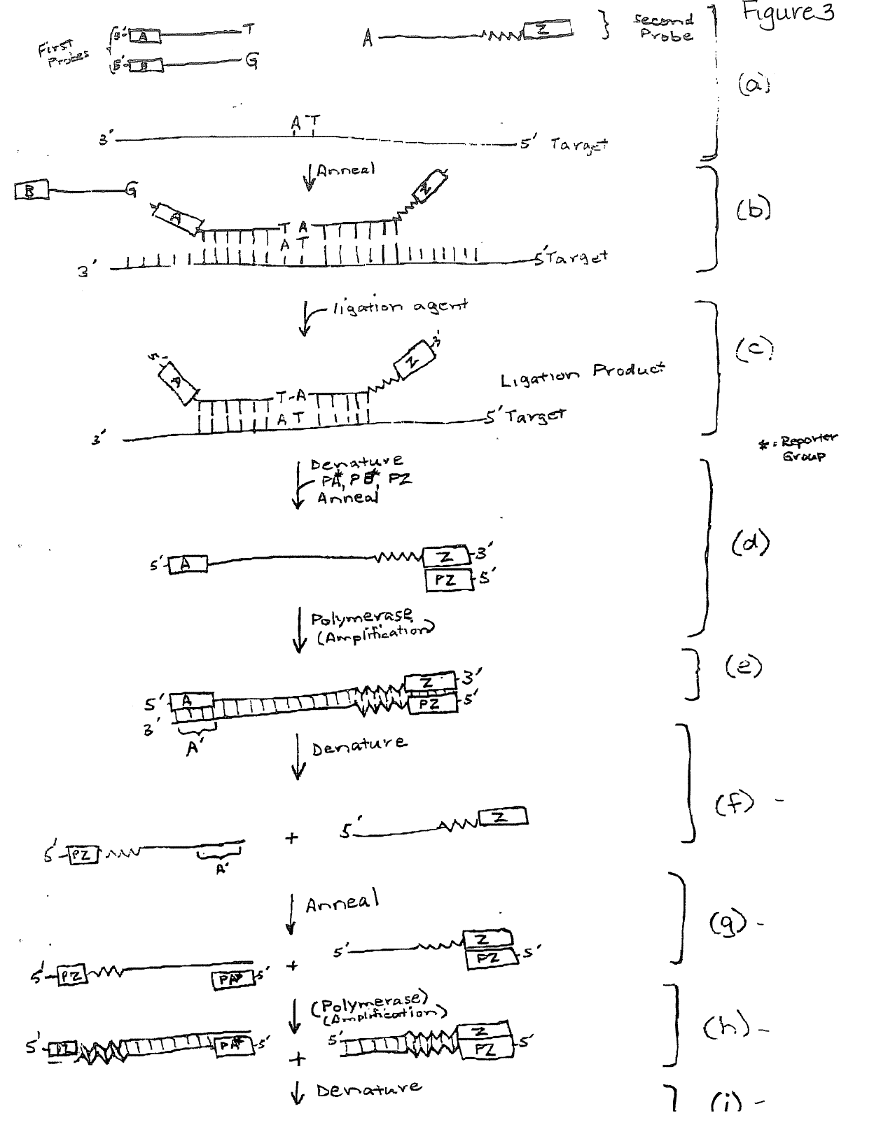

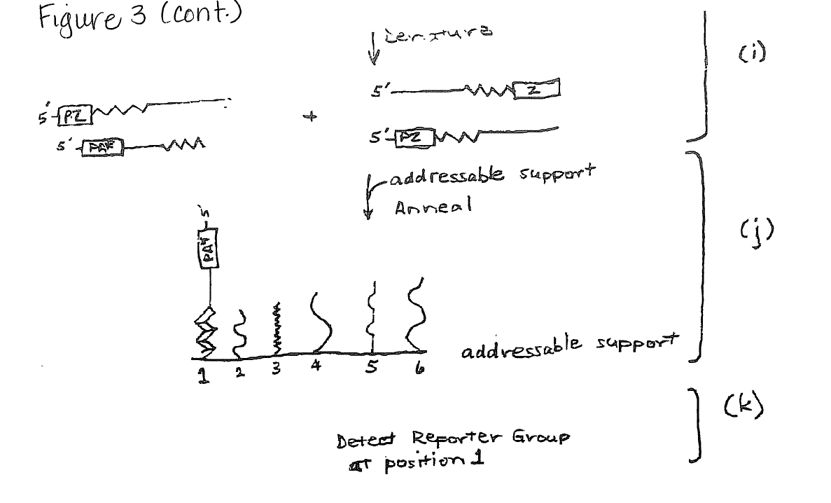

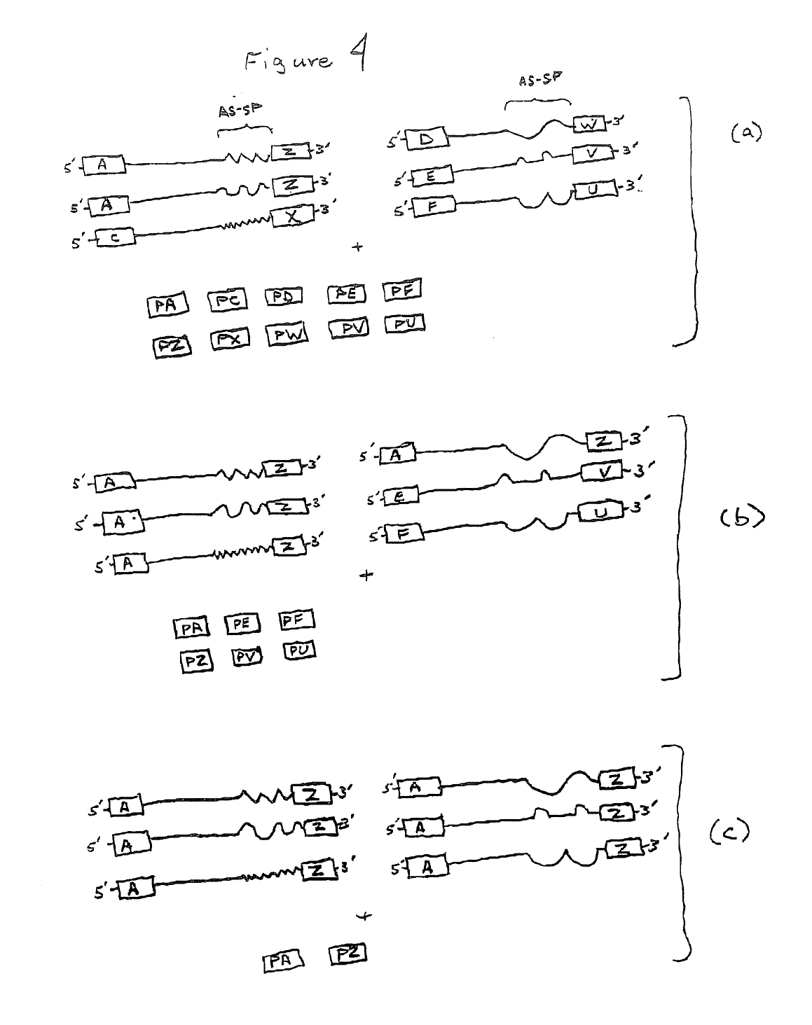

A “target” or “target sequence” according to the present invention comprises a specific nucleic acid sequence, the presence or absence of which is to be detected. The person of ordinary skill will appreciate that while the target sequence is generally described as a single-stranded molecule, the opposing strand of a double-stranded molecule comprises a complementary sequence that may also be used as a target. In certain embodiments, a target sequence comprises an upstream or 5′ region, a downstream or 3′ region, and a “pivotal nucleotide” located between the upstream region and the downstream region (see, e.g., FIG. 1). The pivotal nucleotide is the nucleotide being detected by the probe set and may represent, for example, without limitation, a single polymorphic nucleotide in a multiallelic target locus.

Reagents

Probes, according to the present invention, are oligonucleotides that comprise a target-specific portion that is designed to hybridize in a sequence-specific manner with a complementary region on a selected target sequence. (see, e.g., FIG. 1). A probe may further comprise a primer-specific portion and an addressable support-specific portion.

In at least one probe of a probe set, the addressable support-specific portion is located between the target-specific portion and the primer-specific portion (see, e.g., probe Z in FIG. 1). The addressable support-specific portion may overlap with the target-specific portion or the primer-specific portion, or both. The probe's addressable support-specific portion comprises the sequence that is the same as, or complementary to, a portion of a capture oligonucleotide sequence located on an addressable support. Alternatively, the probe's addressable support-specific portion comprises a mobility modifier that allows detection of the ligation or amplification products based on its location at a particular mobility address due to a mobility detection process, such as, but without limitation, electrophoresis. In one variation, each addressable-support specific portion is complementary to a particular mobility-modifier comprising a tag complement for selectively binding to the addressable support-specific portion of the amplification product, and a tail for effecting a particular mobility in a mobility-dependent analysis technique, e.g., electrophoresis, see, e.g., U.S. patent application Ser. No. 09/522,640, filed Mar. 15, 1999. Preferably, the probe's addressable support-specific portion is not complementary with the target or primer sequences.

The sequence-specific portions of the probes are of sufficient length to permit specific annealing to complementary sequences in primers and targets. The preferred length of the addressable support-specific portion and target-specific portion are 12 to 35 nucleotides. Detailed descriptions of probe design that provide for sequence-specific annealing can be found, among other places, in Diffenbach and Dveksler, PCR Primer, A Laboratory Manual, Cold Spring Harbor Press, 1995, and Kwok et al. (Nucl. Acid Res. 18:999-1005, 1990).

A probe set according to the present invention comprises at least one first probe and at least one second probe that adjacently hybridize to the same target sequence. The first probe in each probe set is designed to hybridize with the downstream region of the target sequence in a sequence-specific manner (see, e.g., probe A in FIG. 1). The second probe in the probe set is designed to hybridize with the upstream region of the target sequence in a sequence-specific manner (see, e.g., probe Z in FIG. 1). The sequence-specific portions of the probes are of sufficient length to permit specific annealing with complementary sequences in targets and primers, as appropriate. In certain embodiments of the invention, both the at least one first probe and the at least one second probe in a probe set further comprise addressable support-specific portions. Preferably, these addressable support-specific portions are not complementary with each other.

Under appropriate conditions, adjacently hybridized probes may be ligated together to form a ligation product, provided that they comprise appropriate reactive groups, for example, without limitation, a free 3′-hydroxyl or 5′-phosphate group. Some probe sets may comprise more than one first probe or more than one second probe to allow sequence discrimination between target sequences that differ by one or more nucleotides (see, e.g., FIG. 2).

According to certain embodiments of the invention, a target-specific probe set is designed so that the target-specific portion of the first probe will hybridize with the downstream target region (see, e.g., probe A in FIG. 1) and the target-specific portion of the second probe will hybridize with the upstream target region (see, e.g., probe Z in FIG. 1). A nucleotide base complementary to the pivotal nucleotide, the “pivotal complement,” is present on the proximal end of either the first probe or the second probe of the target-specific probe set (see, e.g., 3′ end of A in FIG. 1).

When the first and second probes of the probe set are hybridized to the appropriate upstream and downstream target regions, and the pivotal complement is base-paired with the pivotal nucleotide on the target sequence, the hybridized first and second probes may be ligated together to form a ligation product (see, e.g., FIG. 2(b)-(c)). A mismatched base at the pivotal nucleotide, however, interferes with ligation, even if both probes are otherwise fully hybridized to their respective target regions. Thus, highly related sequences that differ by as little as a single nucleotide can be distinguished.

For example, according to certain embodiments, one can distinguish the two potential alleles in a biallelic locus as follows. One can combine a probe set comprising two first probes, differing in their primer-specific portions and their pivotal complement (see, e.g., probes A and B in FIG. 2(a)), one second probe (see, e.g., probe Z in FIG. 2(a)), and the sample containing the target. All three probes will hybridize with the target sequence under appropriate conditions (see, e.g., FIG. 2(b)). Only the first probe with the hybridized pivotal complement, however, will be ligated with the hybridized second probe (see, e.g., FIG. 2(c)). Thus, if only one allele is present in the sample, only one ligation product for that target will be generated (see, e.g., ligation product A-Z in FIG. 2(d)). Both ligation products would be formed in a sample from a heterozygous individual.

Further, in certain embodiments, probe sets do not comprise a pivotal complement at the terminus of the first or the second probe. Rather, the target nucleotide or nucleotides to be detected are located within either the 5′ or 3′ target region. Probes with target-specific portions that are fully complementary with their respective target regions will hybridize under high stringency conditions. Probes with one or more mismatched bases in the target-specific portion, by contrast, will not hybridize to their respective target region. Both the first probe and the second probe must be hybridized to the target for a ligation product to be generated. The nucleotides to be detected may be both pivotal or internal.

In certain embodiments, the first probes and second probes in a probe set are designed with similar melting temperatures (Tₘ). Where a probe includes a pivotal complement, preferably, the Tₘ for the probe(s) comprising the pivotal complement(s) of the target pivotal nucleotide sought will be approximately 4-6° C. lower than the other probe(s) that do not contain the pivotal complement in the probe set. The probe comprising the pivotal complement(s) will also preferably be designed with a Tₘ near the ligation temperature. Thus, a probe with a mismatched nucleotide will more readily dissociate from the target at the ligation temperature. The ligation temperature, therefore, provides another way to discriminate between, for example, multiple potential alleles in the target.

Primers according to the present invention refer to oligonucleotides that are designed to hybridize with the primer-specific portion of probes, ligation products, or amplification products in a sequence-specific manner, and serve as primers for amplification reactions.

The criteria for designing sequence-specific primers and probes are well known to persons of ordinary skill in the art. Detailed descriptions of primer design that provide for sequence-specific annealing can be found, among other places, in Diffenbach and Dveksler, PCR Primer, A Laboratory Manual, Cold Spring Harbor Press, 1995, and Kwok et al. (Nucl. Acid Res. 18:999-1005, 1990). The sequence-specific portions of the primers are of sufficient length to permit specific annealing to complementary sequences in ligation products and amplification products, as appropriate.

According to certain embodiments, the primer sets according to the present invention comprise at least one first primer and at least one second primer (see, e.g., FIG. 3(d)-(g)). The first primer of a primer set is designed to hybridize with the complement of the 5′ primer-specific portion of a ligation or an amplification product in a sequence-specific manner (see, e.g., primer PA in FIG. 3(g)). The second primer in that primer set is designed to hybridize with a 3′ primer-specific portion of the same ligation or amplification product in a sequence-specific manner (see, e.g., primer PZ in FIGS. 3(d) and (g)). In certain embodiments, at least one primer of the primer set further comprises a reporter group. Preferred reporter groups are fluorescent dyes attached to a nucleotide(s) in the primer (see, e.g., L. Kricka, Nonisotopic DNA Probe Techniques, Academic Press, San Diego, Calif. (1992)). Preferably, the reporter group is attached to the primer in such a way as to not to interfere with sequence-specific hybridization or amplification.

A ligation agent according to the present invention may comprise any number of enzymatic or chemical (i.e., non-enzymatic) agents. For example, ligase is an enzymatic ligation agent that, under appropriate conditions, forms phosphodiester bonds between the 3′-OH and the 5′-phosphate of adjacent nucleotides in DNA or RNA molecules, or hybrids. Temperature sensitive ligases, include, but are not limited to, bacteriophage T4 ligase and E. coli ligase. Thermostable ligases include, but are not limited to, Taq ligase, Tth ligase, and Pfu ligase. Thermostable ligase may be obtained from thermophilic or hyperthermophilic organisms.

Chemical ligation agents include, without limitation, activating, condensing, and reducing agents, such as carbodiimide, cyanogen bromide (BrCN), N-cyanoimidazole, imidazole, 1-methylimidazole/carbodiimide/cystamine, dithiothreitol (DTT) and ultraviolet light. Autoligation, i.e., spontaneous ligation in the absence of a ligating agent, is also within the scope of the invention. Detailed protocols for chemical ligation methods and descriptions of appropriate reactive groups can be found, among other places, in Xu et al., Nucleic Acid Res., 27:875-81 (1999); Gryaznov and Letsinger, Nucleic Acid Res. 21:1403-08 (1993); Gryaznov et al., Nucleic Acid Res. 22:2366-69 (1994); Kanaya and Yanagawa, Biochemistry 25:7423-30 (1986); Luebke and Dervan, Nucleic Acids Res. 20:3005-09 (1992); Sievers and von Kiedrowski, Nature 369:221-24 (1994); Liu and Taylor, Nucleic Acids Res. 26:3300-04 (1999); Wang and Kool, Nucleic Acids Res. 22:2326-33 (1994); Purmal et al., Nucleic Acids Res. 20:3713-19 (1992); Ashley and Kushlan, Biochemistry 30:2927-33 (1991); Chu and Orgel, Nucleic Acids Res. 16:3671-91 (1988); Sokolova et al., FEBS Letters 232:153-55 (1988); Naylor and Gilham, Biochemistry 5:2722-28 (1966); and U.S. Pat. No. 5,476,930.

A support or addressable support according to the present invention comprises a support such as a microarray, a microtiter plate, a membrane, beads, including, without limitation, coated or uncoated particles comprising magnetic and paramagnetic material, polyacrylamide, polysaccharide, plastic, and the like, that further comprise bound or immobilized spatially addressable oligonucleotide capture sequence(s), specific ligands, or the like.

Such supports may have a wide variety of geometrys and configurations, and be fabricated using any one of a number of different known fabrication techniques. Exemplary fabrication techniques include, but are not limited to, in situ synthesis techniques, e.g., Southern U.S. Pat. No. 5,436,327 and related patents; light-directed in situ synthesis techniques, e.g., Fodor et al. U.S. Pat. No. 5,744,305 and related patents; robotic spotting techniques, e.g., Cheung et al., Nature Genetics, 21: 15-19 (1999), Brown et al., U.S. Pat. No. 5,807,522, Cantor, U.S. Pat. No. 5,631,134, or Drmanac, U.S. Pat. No. 6,025,136; or arrays of beads having oligonucleotides attached thereto, e.g., Walt, U.S. Pat. No. 6,023,540. Methods used to perform the hybridization process used with the supports are well known and will vary depending upon the nature of the support bound capture nucleic acid and the nucleic acid in solution, e.g., Bowtell, Nature Genetics, 21: 25-32 (1999); Brown and Botstein, Nature Genetics, 21: 33-37 (1999).

Methods

A target nucleic acid sequence for use with the present invention may be derived from any living, or once living, organism, including but not limited to prokaryote, eukaryote, plant, animal, and virus. The target nucleic acid sequence may originate from a nucleus of a cell, e.g., genomic DNA, or may be extranuclear nucleic acid, e.g., plasmid, mitrochondrial nucleic acid, various RNAs, and the like. The target nucleic acid sequence may be first reverse-transcribed into cDNA if the target nucleic acid is RNA. Furthermore, the target nucleic acid sequence may be present in a double stranded or single stranded form.

A variety of methods are available for obtaining a target nucleic acid sequence for use with the compositions and methods of the present invention. When the target nucleic acid sequence is obtained through isolation from a biological matrix, preferred isolation techniques include (1) organic extraction followed by ethanol precipitation, e.g., using a phenol/chloroform organic reagent (e.g., Ausubel et al., eds., Current Protocols in Molecular Biology Volume 1, Chapter 2, Section I, John Wiley & Sons, New York (1 993)), preferably using an automated DNA extractor, e.g., the Model 341 DNA Extractor available from PE Applied Biosystems (Foster City, Calif.); (2) stationary phase adsorption methods (e.g., Boom et al., U.S. Pat. No. 5,234,809; Walsh et al., Biotechniques 10(4): 506-513 (1991)); and (3) salt-induced DNA precipitation methods (e.g., Miller et al., Nucleic Acids Research, 16(3): 9-10 (1988)), such precipitation methods being typically referred to as “salting-out” methods. Optimally, each of the above isolation methods is preceded by an enzyme digestion step to help eliminate unwanted protein from the sample, e.g., digestion with proteinase K, or other like proteases.

Ligation according to the present invention comprises any enzymatic or chemical process wherein an internucleotide linkage is formed between the opposing ends of nucleic acid sequences that are adjacently hybridized to a template. Additionally, the opposing ends of the annealed nucleic acid sequences must be suitable for ligation (suitability for ligation is a function of the ligation method employed). The internucleotide linkage may include, but is not limited to, phosphodiester bond formation. Such bond formation may include, without limitation, those created enzymatically by a DNA or RNA ligase, such as bacteriophage T4 DNA ligase, T4 RNA ligase, Thermus thermophilus (Tth) ligase, Thermus aquaticus (Taq) ligase, or Pyrococcus furiosus (Pfu) ligase. Other internucleotide linkages include, without limitation, covalent bond formation between appropriate reactive groups such as between an α-haloacyl group and a phosphothioate group to form a thiophosphorylacetylamino group, a phosphorothioate a tosylate or iodide group to form a 5′-phosphorothioester, and pyrophosphate linkages.

Chemical ligation may, under appropriate conditions, occur spontaneously such as by autoligation. Alternatively, “activating” or reducing agents may be used. Examples of activating and reducing agents include, without limitation, carbodiimide, cyanogen bromide (BrCN), imidazole, 1-methylimidazole/carbodiimide/cystamine, N-cyanoimidazole, dithiothreitol (DTT) and ultraviolet light.

Ligation generally comprises at least one cycle of ligation, i.e., the sequential procedures of: hybridizing the target-specific portions of a first probe and a second probe, that are suitable for ligation, to their respective complementary target regions; ligating the 3′ end of the first probe with the 5′ end of the second probe to form a ligation product; and denaturing the nucleic acid duplex to separate the ligation product from the target strand. The cycle may or may not be repeated. For example, without limitation, by thermocycling the ligation reaction to linearly increase the amount of ligation product.

Also within the scope of the invention are ligation techniques such as gap-filling ligation, including, without limitation, gap-filling OLA and LCR, bridging oligonucleotide ligation, and correction ligation. Descriptions of these techniques can be found, among other places, in U.S. Pat. No. 5,185,243, published European Patent Applications EP 320308 and EP 439182, and published PCT Patent Application WO 90/01069.

When used in the context of the present invention, “suitable for ligation” refers to at least one first probe and at least one second probe, each comprising an appropriately reactive group. Exemplary reactive groups include, but are not limited to, a free hydroxyl group on the 3′ end of the first probe and a free phosphate group on the 5′ end of the second probe, phosphorothioate and tosylate or iodide, esters and hydrazide, RC(O)S⁻, haloalkyl, RCH₂S and α-haloacyl, thiophosphoryl and bromoacetoamido groups, and S-pivaloyloxymethyl-4-thiothymidine. Additionally, in preferred embodiments, the first and second probes are hybridized to the target such that the 3′ end of the first probe and the 5′ end of the second probe are immediately adjacent to allow ligation.

Purifying the ligation product according to the present invention comprises any process that removes at least some unligated probes, target DNA, enzymes or accessory agents from the ligation reaction mixture following at least one cycle of ligation. Such processes include, but are not limited to, molecular weight/size exclusion processes, e.g., gel filtration chromatography or dialysis, sequence-specific hybridization-based pullout methods, affinity capture techniques, precipitation, adsorption, or other nucleic acid purification techniques. The skilled artisan will appreciate that purifying the ligation product prior to amplification reduces the quantity of primers needed to amplify the ligation product, thus reducing the cost of detecting a target sequence. Also, purifying the ligation product prior to amplification decreases possible side reactions during amplification and reduces competition from unligated probes during hybridization.

Hybridization-based pullout (HBP) according to the present invention comprises a process wherein a nucleotide sequence complementary to at least a portion of one probe, preferably the primer-specific portion, is bound or immobilized to a solid or particulate pullout support (see, e.g., U.S. patent application Ser. No. 08/873,437 to O'Neill et al., filed Jun. 12, 1997). The ligation reaction mixture comprising the ligation product, target sequences, and unligated probes plus buffer components is exposed to the pullout support. The ligation product, under appropriate conditions, hybridizes with the support-bound sequences. The unbound components of the ligation reaction mixture are removed, purifying the ligation products from those ligation reaction mixture components that do not contain sequences complementary to the sequence on the pullout support. One subsequently removes the purified ligation products from the support and combines it with at least one primer set to form a first amplification reaction mixture. The skilled artisan will appreciate that additional cycles of HBP using different complementary sequences on the pullout support will remove all or substantially all of the unligated probes, further purifying the ligation product.

Amplification according to the present invention encompasses a broad range of techniques for amplifying nucleic acid sequences, either linearly or exponentially. Examples of such techniques include, but are not limited to, PCR or any other method employing a primer extension step. Amplification methods may comprise thermal-cycling or may be performed isothermally.

Amplification methods generally comprise at least one cycle of amplification, i.e., the sequential procedures of: hybridizing primers to primer-specific portions of the ligation product or template; synthesizing a strand of nucleotides in a template-dependent manner using a polymerase; and denaturing the newly-formed nucleic acid duplex to separate the strands. The cycle may or may not be repeated.

Primer extension according to the present invention is an amplification process comprising elongating a primer that is annealed to a template in the 5′ to 3′ direction using a template-dependent polymerase. According to certain embodiments, with appropriate buffers, salts, pH, temperature, and nucleotide triphosphates, including analogs and derivatives thereof, a template dependent polymerase incorporates nucleotides complementary to the template strand starting at the 3′-end of an annealed primer, to generate a complementary strand.

According to the present invention, detecting comprises a process for identifying the presence or absence of a particular amplified ligation product that is hybridized to an addressable support or occupying a particular mobility address. For example, when the addressable support-specific portion of an amplification product, or its complement, specifically hybridizes to the capture sequence on the addressable support, the hybridized sequence can be detected provided that a reporter group is present. Typically, the reporter group provides an emission that is detectable or otherwise identifiable in the detection step. The type of detection process used will depend on the nature of the reporter group to be detected. In a particularly preferred detection step used in combination with a fluorescent reporter group, the fluorescent reporter group is detected using laser-excited fluorescent detection.

Generating a single-stranded sequence for hybridization according to the present invention comprises a process for creating single-stranded nucleic acid molecules, or regions within molecules, to facilitate hybridization with single-stranded capture sequences on an addressable support. Processes for generating single-stranded sequence for hybridization include, without limitation, denaturing double-stranded nucleic acid molecules by heating or using chemical denaturants; limited exonuclease digestion of double-stranded nucleic molecules; asymmetric PCR; and single primer amplification or primer extension. Detailed descriptions of such processes can be found, among other places, in Current Protocols in Molecular Biology, John Wiley & Sons, Inc. (1995 and supplements), Sambrook et al., Molecular Cloning, A Laboratory Manual, Cold Spring Harbor Press (1989).

Asymmetric PCR according to the present invention comprises an amplification reaction mixture with an excess of one primer (relative to the other primer in the primer set). Consequently, when the ligation product is amplified, an excess of one strand of the amplification product (relative to its complement) is generated. The single-stranded amplification product may then be hybridized directly with the support-bound capture oligonucleotides.

Asymmetric reamplification according to the present invention comprises generating single-stranded amplification product in a second amplification process. Generally, the double-stranded amplification product of the first amplification process serves as the amplification target in the asymmetric reamplification process. Unlike the first amplification process, however, the second amplification reaction mixture contains only the at least one first primer, or the at least one second primer of a primer set, but not both. The primer in the second amplification reaction mixture comprises a reporter group so that the single-stranded second amplification product is labeled and may be detected when hybridized to the capture oligonucleotides on the addressable support or when occupying a particular mobility address.

Separating by molecular weight or length or mobility according to the present invention is used in the broad sense. Any method that allows a mixture of two or more nucleic acid sequences to be distinguished based on the mobility, molecular weight, or nucleotide length of a particular sequence are within the scope of the invention. Exemplary procedures include, without limitation, electrophoresis, HPLC, mass spectroscopy including MALDI-TOF, and gel filtration.

### Exemplary Embodiments of the Invention

The present invention is directed to methods, reagents, and kits for detecting target nucleic acid sequences in a sample, using coupled ligation and amplification reactions in which (i) a single-stranded addressable support-specific region of the amplified products are detected by hybridization to an addressable support, or (ii) the amplification product is detected at a particular mobility address.

In certain embodiments, for each target nucleic acid sequence to be detected, a probe set, comprising at least one first probe and at least one second probe, is combined with the sample and optionally, a ligation agent, to form a ligation reaction mixture. The first and second probes in each probe set are designed to be complementary to the sequences immediately flanking the pivotal nucleotide of the target sequence (see, e.g., probes A, B, and Z in FIG. 3(a)). Either the at least one first probe or the at least one second probe of a probe set, but not both, will comprise the pivotal complement (see, e.g., probe A of FIG. 3(a)). When the target sequence is present in the sample, the first and second probes will hybridize, under appropriate conditions, to adjacent regions on the target (see, e.g., FIG. 3(b)). When the pivotal complement is base-paired in the presence of an appropriate ligation agent, two adjacently hybridized probes may be ligated together to form a ligation product (see, e.g., Fig Z(c)).

The ligation reaction mixture (in the appropriate salts, buffers, and nucleotide triphosphates) is then combined with at least one primer set and a polymerase to form a first amplification reaction mixture (see, e.g., FIG. 3(d)). In the first amplification cycle, the second primer, comprising a sequence complementary to the 3′ primer-specific portion of the ligation product, hybridizes with the ligation product and is extended in a template-dependent fashion to create a double-stranded molecule comprising the ligation product and its complement (see, e.g., FIG. 3(d)-(e)). When the ligation product exists as a double-stranded molecule, subsequent amplification cycles may exponentially amplify this molecule (see, e.g., FIG. 3(d)-(h)). In FIG. 3, for example, primers PA* and PB* include different reporter groups. Thus, amplification products resulting from incorporation of these primers will include a reporter group specific for the particular pivotal nucleotide that is included in the original target sequence. Certain embodiments of the invention further comprise a second amplification procedure.

Following at least one amplification cycle, the addressable support-specific portions of the amplification products are specifically hybridized with capture oligonucleotides on an addressable support (see, e.g., FIG. 3(i)-(j). The presence of a particular target sequence in the sample is determined by detecting a hybridized amplification product on the support (see, e.g., FIG. 3(k)). As shown in FIG. 3, for example, according to certain embodiments, one can detect the presence of a particular pivotal nucleotide depending on the reporter group detected on the support.

The addressable support specific portion of the amplification product is typically single-stranded in order to hybridize with capture oligonucleotides on the addressable support. In certain embodiments, a single-stranded amplification product is synthesized by, for example, without limitation, asymmetric PCR, primer extension, and asymmetric reamplification.

In an exemplary embodiment of asymmetric PCR, the amplification reaction mixture is prepared as described in Example 1 D below except that for each primer set, either the at least one first primer, or the at least one second primer of a primer set, but not both, are added in excess. Thus the excess primer to limiting primer ratio may be approximately 100:1, respectively. The ideal amounts of the primers should be determined empirically, but will generally range from about 0.2 to 1 pmol for the limiting primer, and from about 10 to 30 pmol for the primer in excess. Empirically, the concentration of one primer in the primer set is typically kept below 1 pmol per 100 μl of amplification reaction mixture.

Since both primers are initially present in substantial excess at the beginning of the PCR reaction both strands are exponentially amplified. Prior to completing all of the cycles of amplification, however, the limiting primer is exhausted. During the subsequent cycles of amplification only one strand is amplified.

After approximately 40 to 45 cycles of amplification are performed, the amplification process is completed with a long extension step. The limiting primer is typically exhausted by the 25^{th} cycle of amplification. During subsequent cycles of amplification only one strand of the amplification product is produced due to the presence of only one primer of the primer set. At the completion of the amplification process the reaction mixture contains a substantial amount of single-stranded amplification product that can be hybridized directly with capture oligonucleotides on the addressable support.

In one exemplary asymmetric reamplification protocol the air-dried first amplification mixture containing double-stranded amplification product from Example 1D below, is resuspended in 30 μl of 0.1× TE buffer, pH 8.0. The second amplification reaction mixture is prepared by combining two microliters of the resuspended amplification product in a 0.2 ml MicroAmp reaction tube with 9 μl sterile filtered deionized water, 18 μl AmpliTaq Gold mix (PE Biosystems, Foster City, Calif.), and 20-40 pmol of either the at least one first primer or the at least one second primer suspended in 1 μl 1× TE buffer. Either the at least one first primer, the at least one second primer, or both are labeled.

The tubes are heated to 95° C. for 12 minutes, then cycled for ten cycles of (94° C. for 15 seconds, 60° C. for 15 seconds, and 72° C. for 30 seconds), followed by twenty-five cycles of (89° C. for 15 seconds, 53° C. for 15 seconds, and 72° C. for 30 seconds), and then 45 minutes at 60° C. The second amplification reaction mixture, containing single-stranded amplification product, is then cooled to 4° C.

Unincorporated PCR primers are removed from the reaction mixture as follows. To each 30 μl amplification reaction mixture 0.34 μl of glycogen (10 mg/ml), 3.09 μl 3 M sodium acetate buffer, pH 5, and 20.6 μl absolute isopropanol are added. The tubes are mixed by vortexing and incubated at room temperature for ten minutes followed by centrifugation at 14,000 rpm for 10-15 minutes in a Beckman Model 18 microfuge.

Supernatants are removed from the labeled amplification product pellets. Each pellet is washed with 50 μl of 70% ethanol with vortexing. The washed amplification products are centrifuged at 14,000 rpm for 5 minutes in a Beckman Model 18 microfuge and the supernatant is removed. The pellets are washed again using 50 μl anhydrous ethanol, vortexed, and centrifuged at 14,000 rpm for 5 minutes, as before. The pellets are air-dried. The dried pellets may be stored at 4° C. prior to hybridization.

In other embodiments, a double-stranded amplification product is generated and subsequently converted into single-stranded sequences. Processes for converting double-stranded nucleic acid into single-stranded sequences include, without limitation, heat denaturation, chemical denaturation, and exonuclease digestion. Detailed protocols for synthesizing single-stranded nucleic acid molecules or converting double-stranded nucleic acid into single-stranded sequences can be found, among other places, in Current Protocols in Molecular Biology, John Wiley & Sons (1995 and supplements); Molecular Cloning, A Laboratory Manual, Cold Spring Harbor Press (1989); and the Novagen Strandase™ product insert.

The skilled artisan will appreciate, however, that when a single-stranded sequence is generated by denaturing a double-stranded sequence, the complementary single-stranded sequences may renature during the support hybridization process. Thus, decreasing the number of single-stranded sequences available for hybridization with the support-bound capture oligonucleotides.

An exemplary nuclease digestion protocol is as follows. The air-dried first amplification product from Example 1D below is resuspended in 10 μl sterile water. Eight microliters of the resuspended amplification product is combined with 1 μl Strandase buffer (Novagen, Madison, Wis.), and 1 μl exonuclease (5 units/μl) in a 0.2 ml MicroAmp reaction tube. The tube is incubated for 20 minutes at 37° C. and the reaction stopped by heating for an additional 10 minutes at 75° C. The nuclease digestion mixture will contain single-stranded or substantially single-stranded first amplification products suitable for hybridization with the capture oligonucleotides on an addressable support.

The skilled artisan will understand that exonucleases, for example, without limitation, λ exonuclease, digest one strand of a double-stranded molecule from a 5′ phosphorylated end. Thus the first amplification product typically comprises a suitable template for nuclease digestion. Suitable templates can be generated during the first amplification process using phosphorylated primers as appropriate. That is, the strand of the amplification product that is to be hybridized with the support will not comprise a primer that is phosphorylated at the 5′-end, while the complementary strand will comprise a 5′ phosphorylated primer. Thus, the complementary strand of the amplification product will be digested by the exonuclease, generating a single-stranded amplification product that is suitable for hybridization.

According to certain embodiments, the novel probes of the present invention comprise a target-specific portion, an addressable support-specific portion, and a primer-specific portion (see, e.g., probe Z of FIG. 1). The probe's target-specific portion is designed to specifically hybridize with a complementary region of the target sequence. The addressable support-specific portion is located between the primer-specific portion and the target-specific portion (see, e.g., probe Z of FIG. 1). Preferably, the probe's addressable support-specific portion is not complementary with the target or primer sequences. The addressable support-specific portion, or its complement, is designed to specifically hybridize with a unique capture oligonucleotide on an addressable support or to have a mobility such that it is located at a particular mobility address during or after appropriate procedures, such as electrophoresis.

In certain embodiments, the 5′ primer-specific portions of at least two different ligation products comprise a sequence that is the same as at least a portion of one first primer in the reaction mixture (see, e.g., PA in FIG. 4(a)). Similarly, at least two different ligation products in a reaction mixture comprise a 3′ primer-specific portion that is complementary to at least a portion of one second primer (in certain embodiments, see, e.g., PZ in FIG. 4(a)). More preferably the 5′ primer-specific portions of most ligation products in a reaction mixture comprise a sequence that is the same as the at least one first primer, and the 3′ primer-specific portions of most of the ligation products in a reaction mixture comprise a sequence that is complementary to at least one second primer (see, e.g., primers PA and PZ in FIG. 4(b)). Most preferably the 5′ primer-specific portions of all ligation products in a reaction mixture comprise a sequence that is the same as the at least one first primer, and the 3′ primer-specific portions of all of the ligation products in a reaction mixture comprise a sequence that is complementary to at least one second primer (see, e.g., primers PA and PZ in FIG. 4(c)).

Such ligation products can be used, for example, but are not limited to, a multiplex reaction to detect multiple target sequences, or multiple potential alleles at multiallelic loci, or combinations of both. According to certain embodiments, a multiplex reaction may include, for example, but is not limited to, six ligation products, each comprising a unique addressable support-specific portion corresponding to different target sequences or alleles or a combination of both (see, e.g., FIG. 4). In FIG. 4(a), the 5′ primer-specific portions of two ligation products (A-Z) comprise a sequence that is the same as at least a portion of one first primer (PA) in the reaction mixture. The 3′ primer-specific portions of the same two ligation products comprise a sequence that is complementary to at least a portion of one second primer in the reaction mixture. Thus, to exponentially amplify these six ligation products, one uses five primer sets (PA-PZ, PC-PX, PD-PW, PE-PV, and PF-PU).

FIG. 4(b) shows the same six ligation products, except that the 5′ primer-specific portions of most of the ligation products comprise a sequence that is the same as at least a portion of one first primer in the reaction mixture. The 3′ primer-specific portions of most of the ligation products comprise a sequence that is complementary to at least a portion of one second primer in the reaction mixture. To exponentially amplify these six ligation products, three primer sets are used (PA-PZ, PE-PV, and PF-PU).

FIG. 4(c) shows the same six ligation products, except that the 5′ primer-specific portions of all of the ligation products comprise a sequence that is the same as at least a portion of one first primer in the reaction mixture. The 3′ primer-specific portions of all of the ligation products comprise a sequence that is complementary to at least a portion of one second primer in the reaction mixture. To exponentially amplify these six ligation products, only one primer set is used (PA-PZ).

Thus, the same primer set will be used for at least two ligation products in the reaction mixture (see, e.g., primers PA and PZ of FIG. 4(a)). Preferably most ligation products in the reaction mixture will use the same primer set (see, e.g., primers PA and PZ of FIG. 4(b)). Most preferably all of the ligation products in the reaction mixture will use the same primer set (see, e.g., primers PA and PZ of FIG. 4(c)).

The methods of the instant invention, therefore, decrease the number of different primers that must be added to the reaction mixture, reducing the cost and time of target sequence detection. For example, without limitation, in a multiplex reaction designed to detect 100 target sequences, 100 different primer sets are required using certain conventional methods. Since the methods of the present invention permit at least two amplification products and most preferably all of the amplification products to comprise the same primer-specific portions, no more than 99 different primer sets are required, most preferably only 1.

Because only a limited number of primers are required for amplification, the novel methods provided herein allow genomic DNA to be used directly and are more cost-efficient and less time-consuming than conventional methods of detecting known sequences in a sample. Using a limited number of primers may also reduce variation in amplification efficiency and cross-reactivity of the primers.

The skilled artisan will appreciate, however, that in certain embodiments, including, but not limited to, detecting multiple alleles, the ligation reaction mixture may comprise more than one first probe or more than one second probe for each potential allele in a multiallelic target locus. Those methods preferably employ more than one first primer or more than one second primer in a reaction mixture. For example, one first primer for all first alleles to be detected, a different first primer for all second alleles to be detected, another first primer for all third alleles to be detected, and so forth.

The significance of the decrease in the number of primers, and therefore the cost and number of manipulations required, becomes readily apparent when performing genetic screening of an individual for a large number of multiallelic loci. In certain embodiments, one may use, for example, without limitation, a simple screening assay to detect the presence of three biallelic loci (e.g., L1, L2, and L3) in an individual using three probe sets. See, e.g., Table 1 below.

TABLE 1

| | | | | Addressable |
|---|---|---|---|---|
| | | | | Support- |
| | | | | Specific |
| Locus | Allele | Probe Set | Primer Set | Sequence |
| L1 | 1 | A1, Z1 | PA, PZ | 1 |
| | 2 | B1, Z1 | PB, PZ | 2 |
| L2 | 1 | A2, Z2 | PA, PZ | 3 |
| | 2 | B2, Z2 | PB, PZ | 4 |
| L3 | 1 | A3, Z3 | PA, PZ | 5 |
| | 2 | B3, Z3 | PB, PZ | 6 |

For illustration purposes, each of the three probe sets comprise two first probes, for example, A and B, and one second probe, Z. Both first probes, A and B, comprise the same upstream target-specific sequence, but differ at the pivotal complement. The skilled artisan, however, will understand that the probes can be designed with the pivotal complement at any location in either the first probe or the second probe. Additionally, probes comprising multiple pivotal complements are within the scope of the invention.

To distinguish between the two possible alleles in each biallelic locus, probes A and B comprise different 5′ primer-specific sequences. Therefore, two different first primers, PA and PB, hybridize with the complement of the primer-specific portions of probe A and probe B, respectively. A third primer, PZ, hybridizes with the primer-specific portion of probe Z. If the different first primers comprise different reporter groups, the reporter groups can be used to distinguish between the allele-specific ligation products. Thus, in these embodiments three probes A1, B1, and Z1, are used to form the two possible L1 ligation products, wherein A1Z1 is the ligation product of the first L1 allele and B1Z1 is the ligation product of the second L1 allele. Likewise, probes A2, B2, and Z2, are used to form the two possible L2 ligation products. Probe A2 comprises the same primer-specific portion as probe A1, the primer-specific portion of probe B2 is the same as probe B1, and so forth. Thus, as few as three primers, PA, PB, and PZ, could be used in these embodiments. According to these embodiments, the detection of only one label at the capture oligonucleotide or at a particular mobility location would indicate that the sample was obtained from a homozygous individual. Both labels would be detected at the capture oligonucleotide or mobility location if the sample was obtained from a heterozygous individual.

In these embodiments, the number of probes needed to detect any number of target sequences, therefore, is the product of the number of targets to be detected times the number of alleles to be detected per target plus one (i.e., (number of target sequences×[number of alleles+1]). Thus, to detect 3 biallelic sequences, for example, nine probes are needed (3×[2+1]), or as shown in Table 1, (A1, B1, Z1, A2, B2, Z2, A3, B3, and Z3). To detect 4 triallelic sequences 16 probes are needed (4×[3+1]), and so forth.

In these embodiments, to amplify the ligation product of target sequence L1, three primers are needed to address a biallelic locus, PA, complementary to the 5′ primer-specific portion of A1; PB, complementary to the 5′ primer-specific portion of B1; and PZ, complementary to the 3′ primer-specific portion of Z1, respectively. To amplify the ligation product of target sequence L2, using certain conventional methods, three additional primers are required, e.g., PA2, PB2, and PZ2; likewise to amplify target sequence L3, requires yet three more primers, PA3, PB3, and PZ3. Thus, to amplify the ligation products for three biallelic loci potentially present in an individual using certain conventional methodology, would require 9 (3n, where n=3) primers.

In contrast, the methods of the present invention can effectively reduce this number to as few as three amplification primers in this example. Using the present invention, one can use at least two different A probes that comprise the same 5′ primer-specific sequence. More preferably, most of the different A probes comprise the same 5′ primer-specific sequence. Most preferably, all of the different A probes comprise the same 5′ primer-specific sequence. Similarly, at least two, more preferably most, and most preferably all of the different B probes comprise the same 5′ primer-specific sequence. Finally, at least two, more preferably most, and most preferably all of the different Z probes comprise the same 3′ primer-specific sequence. Thus, as few as one A primer, one B primer, and one Z primer can be used to amplify all of ligation products (PA, PB and PZ in Table 1).

In other embodiments, one can use different addressable support-specific sequences to distinguish between the allele-specific ligation products. Thus, for a biallelic locus, for example, but without limitation, the same first labeled primer can be used to hybridize with the complement of either probe A or probe B. A second primer, PZ, hybridizes with the primer-specific portion of probe Z. Thus, as few as two primers could be used in these embodiments. According to these embodiments, the detection of only a single labeled amplification product hybridized to its respective capture oligonucleotide or at a mobility location would indicate that the sample was obtained from a homozygous individual. If the sample was obtained from a heterozygous individual, both amplification products would hybridize with their respective capture oligonucleotides or be detected at appropriate mobility locations.

According to the present invention, as few as two or three “universal” primers, can be used to amplify an infinite number of ligation or amplification products, since the probes may be designed to share primer-specific portions but comprise different addressable support-specific portions.

Rather than the nine primers required to detect all potential alleles in three biallelic loci, using certain conventional methodology (e.g., PA1, PB1, PZ1, PA2, PB2, PZ2, PA3, PB3, and PZ3), the methods of the present invention can use as few as three primers (PA, PB, and PZ, as shown in Table 1). A sample containing 100 possible biallelic loci would require 200 primers in certain conventional detection methods, yet only 3 universal primers can be used in the instant methods. This dramatic decrease in the number of required amplification primers is possible since at least one probe in each probe set has the addressable support-specific sequence located between the primer-specific portion and the target-specific portion.

In certain embodiments, different alleles in a multiallelic locus are differentiated using primers with different reporter groups. For example, but without limitation, if the first allele is present in the sample, the ligation product will comprise primer-specific portion A. If the second allele is present in the sample, the ligation product will comprise primer-specific portion B. In certain embodiments, primer PA, complementary to portion A, comprises a green reporter group, while primer PB, complementary to portion B, comprises a red reporter group. The two alleles are differentiated by detecting either a green or a red reporter group hybridized via the addressable support-specific portion to the support at a spatially addressable position or at a mobility location. Both the green and the red reporter groups will be detected if the individual is heterozygous for the bialielic target locus.

In other embodiments, different alleles in a multiallelic locus are differentiated using probes with different addressable-support-specific portions. For example, but without limitation, if the first allele is present in the sample, the ligation product will comprise addressable support-specific portion A. If the second allele is present in the sample, the ligation product will comprise addressable support-specific portion B. At least one primer for each ligation product comprises a red reporter group. The two alleles are differentiated by detecting a red reporter group hybridized with the support at one of two spatially addressable positions or mobility locations. The person of ordinary skill will appreciate that three or more alleles at a multiallelic locus can also be differentiated using these methods.

In certain embodiments, different reporter groups and different addressable support-specific sequences are combined to distinguish different targets and/or different alleles.

In yet other embodiments, the at least one first probes and the at least one second probes in a probe set comprise different reporter groups.

In yet other embodiments, different targets and/or different alleles are detected by mobility discrimination using separation techniques such as electrophoresis, mass spectroscopy, or chromatography rather than hybridization to capture oligonucleotides on a support. In these embodiments the probes may comprise addressable support-specific portions of unique identifiable lengths or molecular weights. Alternatively, each addressable support-specific portion is complementary to a particular mobility-modifier comprising a tag complement for selectively binding to the addressable support-specific portion of the amplification product, and a tail for effecting a particular mobility in a mobility-dependent analysis technique, e.g., electrophoresis, e.g., U.S. patent application Ser. No. 09/522,640, filed Mar. 15, 1999. Thus, the amplification products can be separated by molecular weight or length to distinguish the individual amplified sequences. The detection of an amplification product in a particular molecular weight or length bin indicates the presence of the target sequence in the sample. Descriptions of mobility discrimination techniques may be found, among other places, in U.S. Pat. Nos. 5,470,705, 5,514,543, 5,580,732, 5,624,800, and 5,807,682.

In an exemplary protocol, the air-dried amplification pellets of Example 1 D below, comprising amplification products of uniquely identifiable molecular weight, are resuspended in buffer or deionized formamide. The resuspended samples and a molecular weight marker (e.g., GS 500 size standard, PE Biosystems, Foster City, Calif.) are loaded onto an electrophoresis platform (e.g., ABI Prism™ Genetic Analyzer, PE Biosystems, Foster City, Calif.) and electrophoresed (in POP-4 polymer, PE Biosystems, Foster City, Calif.; at 15 kV using a 50 I capillary). The bands are detected and their position relative to the marker is determined. The bands are identified based on their relative electrophoretic mobility, indicating the presence of their respective target sequence in the sample.

Alternatively, each addressable-support specific portion contains a sequence that is complementary to a mobility-modifier comprising a tag complement that is complementary to the addressable support-specific portion of the amplification product, and a tail, for effecting a particular mobility in a mobility-dependent analysis technique, e.g., electrophoresis, such that when the tag complement and the addressable support-specific portion are contacted a stable complex is formed, see, e.g., U.S. patent application Ser. No. 09/522,640 filed Mar. 15, 1999. As used herein, “mobility-dependent analysis technique” means an analysis technique based on differential rates of migration between different analyte species. Exemplary mobility-dependent analysis techniques include electrophoresis, chromatography, mass spectroscopy, sedimentation, e.g., gradient centrifugation, field-flow fractionation, multi-stage extraction techniques and the like.

According to this embodiment of the invention, preferred addressable support-specific portions and tag-complements should form a complex that (1) is stable under conditions typically used in nucleic acid analysis methods, e.g., aqueous, buffered solutions at room temperature; (2) is stable under mild nucleic-acid denaturing conditions; and (3) does not adversely effect a sequence specific binding of a target-specific portion of a probe with a target nucleic acid sequence. In addition, addressable support-specific portions and tag complements of the invention should accommodate sets of distinguishable addressable support-specific portions and tag complements such that a plurality of different amplification products and associated mobility modifiers may be present in the same reaction volume without causing cross-interactions among the addressable support-specific portions, tag complements, target nucleic acid sequence and target-specific portions of the probes. Methods for selecting sets of tag sequences that minimally cross hybridize are described elsewhere (e.g., Brenner and Albrecht, PCT Patent Application No. WO 96/41011).

In a preferred embodiment, the addressable support-specific portions and tag complement each comprise polynucleotides. In a preferred polynucleotide tag complement, the tag complements are rendered non-extendable by a polymerase, e.g., by including sugar modifications such as a 3′-phosphate, a 3′-acetyl, a 2′-3′-dideoxy, a 3′-amino, and a 2′-3′ dehydro.

A particularly preferred addressable support-specific portion and tag complement pair comprises an addressable support-specific portion that is a conventional synthetic polynucleotide, and a tag complement that is PNA. Where the PNA tag complement has been designed to form a triplex structure with a tag, the tag complement may include a “hinge” region in order to facilitate triplex binding between the tag and tag complement. In a more preferred embodiment, addressable support-specific portions and tag complement sequences comprise repeating sequences. Such repeating sequences in the addressable support-specific portions and tag complement are preferred by virtue of their (1) high binding affinity, (2) high binding specificity, and (3) high solubility. A particularly preferred repeating sequence for use as a duplex-forming addressable support-specific portions or tag complement is (CAG)ₙ, where the three base sequence is repeated from about 1 to 10 times (see, e.g., Boffa, et al., PNAS (USA), 92:1901-05 (1995); Wiftung, et al., Biochemistry, 36:7973-79 (1997)). A particularly preferred repeating sequence for use as a triplex-forming addressable support-specific portions or tag complement is (TCC)ₙ.

PNA and PNA/DNA chimera molecules can be synthesized using well known methods on commercially available, automated synthesizers, with commercially available reagents (e.g., Dueholm, et al., J. Org. Chem., 59:5767-73 (1994); Vinayak, et al., Nucleosides & Nucleotides, 16:1653-56 (1997)).

The addressable support-specific portions may comprise all, part, or none of the target-specific portion of the probe. In some embodiments of the invention, the addressable support-specific portions may consist of some or all of the target-specific portion of the probe. In other embodiments of the invention, the addressable support-specific portions do not comprise any portion of the target-specific portion of the probe.

In certain embodiments, the mobility modifier of the present invention comprise a tag complement portion for binding to the addressable support-specific portion of the amplification product, and a tail for effecting a particular mobility in a mobility-dependent analysis technique.

The tail portion of a mobility modifier may be any entity capable of effecting a particular mobility of a amplification product/mobility-modifier complex in a mobility-dependent analysis technique. Preferably, the tail portion of the mobility modifier of the invention should (1) have a low polydispersity in order to effect a well-defined and easily resolved mobility, e.g., Mw/Mn [Should we define Mw and Mn?] less than 1.05; (2) be soluble in an aqueous medium; (3) not adversely affect probe-target hybridization or addressable support-specific portion/tag complement binding; and (4) be available in sets such that members of different sets impart distinguishable mobilities to their associated complexes.

In a particularly preferred embodiment of the present invention, the tail portion of the mobility modifier comprises a polymer. Specifically, the polymer forming the tail may be homopolymer, random copolymer, or block copolymer. Furthermore, the polymer may have a linear, comb, branched, or dendritic architecture. In addition, although the invention is described herein with respect to a single polymer chain attached to an associated mobility modifier at a single point, the invention also contemplates mobility modifiers comprising more than one polymer chain element, where the elements collectively form a tail portion.

Preferred polymers for use in the present invention are hydrophilic, or at least sufficiently hydrophilic when bound to a tag complement to ensure that the tag complement is readily soluble in aqueous medium. Where the mobility-dependent analysis technique is electrophoresis, the polymers are preferably uncharged or have a charge/subunit density that is substantially less than that of the amplification product.

In one preferred embodiment, the polymer is polyethylene oxide (PEO), e.g., formed from one or more hexaethylene oxide (HEO) units, where the HEO units are joined end-to-end to form an unbroken chain of ethylene oxide subunits. Other exemplary embodiments include a chain composed of N 12mer PEO units, and a chain composed of N tetrapeptide units, where N is an adjustable integer (e.g., Grossman et al., U.S. Pat. No. 5,777,096).

Clearly, the synthesis of polymers useful as tail portions of a mobility modifier of the present invention will depend on the nature of the polymer. Methods for preparing suitable polymers generally follow well known polymer subunit synthesis methods. Methods of forming selected-length PEO chains are discussed below. These methods, which involve coupling of defined-size, multi-subunit polymer units to one another, either directly or through charged or uncharged linking groups, are generally applicable to a wide variety of polymers, such as polyethylene oxide, polyglycolic acid, polylactic acid, polyurethane polymers, polypeptides, and oligosaccharides. Such methods of polymer unit coupling are also suitable for synthesizing selected-length copolymers, e.g., copolymers of polyethylene oxide units alternating with polypropylene units. Polypeptides of selected lengths and amino acid composition, either homopolymer or mixed polymer, can be synthesized by standard solid-phase methods (e.g., Fields and Noble, Int. J. Peptide Protein Res., 35: 161-214 (1990)).

In one preferred method for preparing PEO polymer chains having a selected number of HEO units, an HEO unit is protected at one end with dimethoxytrityl (DMT), and activated at its other end with methane sulfonate. The activated HEO is then reacted with a second DMT-protected HEO group to form a DMT-protected HEO dimer. This unit-addition is then carried out successively until a desired PEO chain length is achieved (e.g., Levenson et al., U.S. Pat. No. 4,914,210).

Another particularly preferred polymer for use as a tail portion is PNA. The advantages, properties and synthesis of PNA have been described above. In particular, when used in the context of a mobility-dependent analysis technique comprising an electrophoretic separation in free solution, PNA has the advantageous property of being essentially uncharged.

Coupling of the polymer tails to a polynucleotide tag complement can be carried out by an extension of conventional phosphoramidite polynucleotide synthesis methods, or by other standard coupling methods, e.g., a bis-urethane tolyl-linked polymer chain may be linked to an polynucleotide on a solid support via a phosphoramidite coupling. Alternatively, the polymer chain can be built up on a polynucleotide (or other tag portion) by stepwise addition of polymer-chain units to the polynucleotide, e.g., using standard solid-phase polymer synthesis methods.

As noted above, the tail portion of the mobility modifier imparts a mobility to a amplification product/mobility modifier complex that is distinctive for each different probe/mobility modifier complex. The contribution of the tail to the mobility of the complex will in generally depend on the size of the tail. However, addition of charged groups to the tail, e.g., charged linking groups in the PEO chain, or charged amino acids in a polypeptide chain, can also be used to achieve selected mobility characteristics in the probe/mobility modifier complex. It will also be appreciated that the mobility of a complex may be influenced by the properties of the amplification product itself, e.g., in electrophoresis in a sieving medium, a larger probe will reduce the electrophoretic mobility of the probe/mobility modifier complex.

The tag complement portion of a mobility modifier according to the present invention may be any entity capable of binding to, and forming a complex with, an addressable support-specific portion of an amplification product. Furthermore, the tag-complement portion of the mobility modifier may be attached to the tail portion using conventional means.

When a tag complement is a polynucleotide, e.g., PNA, the tag complement may comprise all, part, or none of the tail portion of the mobility modifier. In some embodiments of the invention, the tag complement may consist of some or all of the tail portion of the mobility modifier. In other embodiments of the invention, the tag complement does not comprise any portion of the tail portion of the mobility modifier. For example, because PNA is uncharged, particularly when using free solution electrophoresis as the mobility-dependent analysis technique, the same PNA oligomer may act as both a tag complement and a tail portion of a mobility modifier.

In a preferred embodiment of the present invention, the tag complement includes a hybridization enhancer, where, as used herein, the term “hybridization enhancer” means moieties that serve to enhance, stabilize, or otherwise positively influence hybridization between two polynucleotides, e.g. intercalators (e.g., U.S. Pat. No. 4,835,263), minor-groove binders (e.g., U.S. Pat. No. 5,801,155), and cross-linking functional groups. The hybridization enhancer may be attached to any portion of a mobility modifier, so long as it is attached to the mobility modifier is such a way as to allow interaction with the addressable support-specific portion/tag complement duplex. However, preferably, the hybridization enhancer is covalently attached to a mobility modifier of the binary composition. A particularly preferred hybridization enhancer for use in the present invention is minor-groove binder, e.g., netropsin, distamycin, and the like.

According to an important feature of the invention, a plurality of amplification product/mobility modifier complexes are resolved via a mobility-dependent analysis technique.

In one embodiment of the invention, amplification product/mobility modifier complexes are resolved (separated) by liquid chromatography. Exemplary stationary phase media for use in the method include reversed-phase media (e.g., C-18 or C-8 solid phases), ion-exchange media (particularly anion-exchange media), and hydrophobic interaction media. In a related embodiment, the amplification product/mobility modifier complexes can be separated by micellar electrokinetic capillary chromatography (MECC).

Reversed-phase chromatography is carried out using an isocratic, or more typically, a linear, curved, or stepped solvent gradient, wherein the level of a nonpolar solvent such as acetonitrile or isopropanol in aqueous solvent is increased during a chromatographic run, causing analytes to elute sequentially according to affinity of each analyte for the solid phase. For separating polynucleotides, an ion-pairing agent (e.g., a tetra-alkylammonium) is typically included in the solvent to mask the charge of phosphate.

The mobility of an amplification product/mobility modifier complex can be varied by using mobility modifiers comprising polymer chains that alter the affinity of the probe for the solid, or stationary, phase. Thus, with reversed phase chromatography, an increased affinity of the amplification product/mobility modifier complexes for the stationary phase can be attained by addition of a moderately hydrophobic tail (e.g., PEO-containing polymers, short polypeptides, and the like) to the mobility modifier. Longer tails impart greater affinity for the solid phase, and thus require higher non-polar solvent concentration for the probe to be eluted (and a longer elution time).

According to a particularly preferred embodiment of the present invention, the amplification product/mobility modifier complexes are resolved by electrophoresis in a sieving or non-sieving matrix. Preferably, the electrophoretic separation is carried out in a capillary tube by capillary electrophoresis (e.g., Capillary Electrophoresis: Theory and Practice, Grossman and Colburn eds., Academic Press (1992)). Preferred sieving matrices which can be used include covalently crosslinked matrices, such as polyacrylamide covalently crosslinked with bis-acrylamide; gel matrices formed with linear polymers (e.g., Madabhushi et al. U.S. Pat. No. 5,552,028); and gel-free sieving media (e.g., Grossman et al., U.S. Pat. No. 5,624,800; Hubert and Slater, Electrophoresis, 16: 2137-2142 (1995); Mayer et al., Analytical Chemistry, 66(10): 1777-1780 (1994)). The electrophoresis medium may contain a nucleic acid denaturant, such as 7M formamide, for maintaining polynucleotides in single stranded form. Suitable capillary electrophoresis instrumentation are commercially available, e.g., the ABI PRISM™ Genetic Analyzer (PE Biosystems, Foster City, Calif.).

The skilled artisan will appreciate that the amplification products can also be separated based on molecular weight and length or mobility by, for example, but without limitation, gel filtration, mass spectroscopy, or HPLC, and detected using appropriate methods.

For each target sequence to be detected a probe set, comprising at least one first probe and at least one second probe, is combined with the sample, and optionally a ligation agent, to form a ligation reaction mixture. Either the at least one first probe or the at least one second probe comprise an addressable support-specific portion, located between the primer-specific portion and the target-specific portion, that is identifiable by molecular weight or length or is complementary to a particular mobility modifier. For example, without limitation, the addressable support-specific portion that corresponds to one target sequence will be 2 nucleotides in length, the addressable support-specific portion that corresponds to second target sequence will be 4 nucleotides in length, the addressable support-specific portion that corresponds to a third target sequence will be 6 nucleotides in length, and so forth. Preferably, the addressable support-specific portion in these embodiments will be 0 to 100 nucleotides long, more preferably 0 to 40 nucleotides long, and most preferably 2 to 36. Preferably the addressable support-specific portions that correspond to a particular target sequence will differ in length from the addressable support-specific portions that correspond to different target sequences by at least two nucleotides.

The first and second probes in each probe set are designed to be complementary to the sequences immediately flanking the pivotal nucleotide of the target sequence. Either the at least one first probe or the at least one second probe of a probe set, but not both, will comprise the pivotal complement. When the target sequence is present in the sample, the first and second probes will hybridize, under appropriate conditions, to adjacent regions on the target. When the pivotal complement is base-paired in the presence of an appropriate ligation agent, two adjacently hybridized probes may be ligated together to form a ligation product. Alternatively, under appropriate conditions, autoligation may occur. The skilled artisan will appreciate that the pivotal nucleotide(s) may be located anywhere in the target sequence and that likewise, the pivotal complement may be located anywhere within the target-specific portion of the probe(s).

The ligation reaction mixture (in the appropriate salts, buffers, and nucleotide triphosphates) is then combined with at least one primer set and a polymerase to form a first amplification reaction mixture. In the first amplification cycle, the second primer, comprising a sequence complementary to the 3′ primer-specific portion of the ligation product, hybridizes with the ligation product and is extended in a template-dependent fashion to create a double-stranded molecule comprising the ligation product and its complement. When the ligation product exists as a double-stranded molecule, subsequent amplification cycles may exponentially amplify this molecule.

The primer set comprises at least one reporter group so that the amplification products resulting from incorporation of these primers will include a reporter group specific for the particular pivotal nucleotide that is included in the original target sequence.

Following at least one amplification cycle, the amplification products are separated based on their molecular weight or length or mobility by, for example, without limitation, gel electrophoresis, HPLC, MALDI-TOF, gel filtration, or mass spectroscopy. The detection of a labeled sequence at a particular mobility address indicates that the sample contains the related target sequence.

The invention also provides kits designed to expedite performing the subject methods. Kits serve to expedite the performance of the methods of interest by assembling two or more components required for carrying out the methods. Kits preferably contain components in pre-measured unit amounts to minimize the need for measurements by end-users. Kits preferably include instructions for performing one or more methods of the invention. Preferably, the kit components are optimized to operate in conjunction with one another.

The kits of the invention may be used to ligate adjacently hybridized probes, to amplify ligation products, to generate single-stranded nucleic acids from double-stranded molecules, or combinations of these processes. The kits of the invention may further comprise additional components such as oligonucleotide triphosphates, nucleotide analogs, reaction buffers, salts, ions, stabilizers, or combinations of these components. Certain kits of the invention comprise reagents for purifying the ligation products, including, without limitation, dialysis membranes, chromatographic compounds, supports, oligonucleotides, or combinations of these reagents.

The invention, having been described above, may be better understood by reference to examples. The following examples are intended for illustration purposes only, and should not be construed as limiting the scope of the invention in any way.

### EXAMPLE 1

### SNP Detection using Coupled Ligation and Amplification

A. Preparation of Genomic Target DNA

Target nucleic acid derived from genomic DNA was prepared by DNAse I digestion as follows. Aliquots of genomic DNA containing 1.6 μl 500 mM Tris-HCl, pH 7.5, 6.4 μl 25 mM MgCl₂, 6.0 μl genomic DNA (300 ng/ml), and 2.0 μl 0.0125 u/μl DNAse I (in 50% glycerol, 50 μl Tris-HCl pH7.5), were incubated at 25° C. for 20 minutes. The enzyme was heat inactivated at 99° C. for 15 minutes and two I of 1× TE (10 mM Tris-HCl, pH 7.5, 1 mM EDTA) were added to adjust the final DNA concentration to 100 ng/μl. These aliquots of fragmented genomic DNA may be stored at minus 20° C.

B. Ligation of Target-Specific Probe Sets

Thirteen target-specific probe sets, shown in Table 2, were designed to detect thirteen different biallelic loci or their complement in a multiplex reaction. The probe sets were prepared on an ABI 3948 DNA synthesizer (PE Biosystems, Foster City, Calif.), using standard phosphoramidite chemistry according to the manufacturer's instructions. The second probes were phosphorylated during synthesis using phospholink chemistry (see, e.g., H. Guzaev et al., Tetrahedron 51:9375-84 (1995)).

The probe sets were ligated together as follows. Two microliters of the fragmented genomic DNA, 4.5 μl sterile filtered, deionized water, 1 μl 100 mM KCl, 1 μl 10× ligase buffer (0.2 M Tris-HCl, pH 7.6 at 25° C., 0.25 M sodium acetate, 0.1 M magnesium acetate, 0.1 M DTT, 0.01 M nicotinamide adenine dinucleotide (NAD), and 1% Triton X-100), and 0.5 μl ligase mix (9.5 μl 1× ligase buffer and 0.5 μl Taq DNA ligase, 40 u/μl) were combined in a 0.2 ml MicroAmp reaction tube (PE Biosystems, Foster City, Calif.). This mixture was vortexed and incubated at room temperature for three minutes. One microliter of target-specific probe mix (24 nM of each probe in 1× TE) was added to the ligation reaction mixture and the reaction mixture was mixed by vortexing.

The tubes were placed in a thermal cycler and subjected to multiple cycles of ligation as follows. The tubes were cycled between 90° C. for 5 seconds and

TABLE 2

| | | | |
|---|---|---|---|
| Locus | First Probes (Tₘ ° C.) | Second Probe (Tₘ ° C.) | TARGET SEQUENCE |

Exemplary Multiplex Probe Sets

| | | | |
|---|---|---|---|
| 03_04 | AACTCTCTCCCAAGAGCGAGGCCAACT | P-ACAACTGGGAAGAGCCGTAAGCGGGA | TCCTCCTGGGAAAATAATCCTGTTGGA |
| | AACCAA (45.8) (SEQ ID NO:1) | CCGTCAGAATCATGTAAAACGACGGCCA | GTTGGGGGCTCTTCCCAGTTGT[T/C]T |
| | CACTCACGCAAACGGGCCAACTAACCA | GT (53.3) (SEQ ID NO:3) | GGTTAGTTGGCCCAGGAAGGGGCAGT |
| | G (45.0) (SEQ ID NO:2) | | CCTGGAGCTGGCGGGTG (SEQ ID |
| | | | NOs:69 and 82) |
| 04_01 | AACTCTCTCCCAAGAGCGATTGGCGAG | P-GAGAGCCAGCTCTGCACAAGCCATCTC | CAGGCACAACCATGGCTGCGTGATGA |
| | TGAGTT (47.7) (SEQ ID NO:6) | CTGTCCACGATGTAAAACGACGGCCAGT | CCCCGCAGAGCTGGCTCTC[A/C]ACTC |
| | CACTCACGCAAACGGGATTGGCGAGTG | (51.0) (SEQ ID NO:8) | ACTCGCCAATTTCAGAGTACAGTGGT |
| | AGTG (48.6) (SEQ ID NO:7) | | GGGGTGCGGTGTGT (SEQ ID NOs:70 |
| | | | and 83) |
| 05_01 | AACTCTCTCCCAAGAGCGATTAGCCTG | P-TAAAGAGAAACTTTGTGCTCCAAGCGT | GAGATTTAGCAGCTTTGTCGTCATGTA |
| | TGGCAA (47.3) (SEQ ID NO:11) | GGTCCACTCCGATGTAAAACGACGGCCA | GCACAAAGTTTCTCTTTA[T/C]TGCCAC |
| | CACTCACGCAAACGGGTTAGCCTGTGG | GT (51.8) (SEQ ID NO:13) | AGGCTAAGAATGCTGAACAGGAAAGG |
| | CAG (46.5) (SEQ ID NO:12) | | CACCAAAGAA (SEQ ID NOs:71 and |
| | | | 84) |
| 06_01 | AACTCTCTCCCAAGAGCGATCGAGGAC | P-GGCCTGTCTGTCCACTCAAGCGATTCC | TTAAATGCCTGTCTCCCCGCGGATCAT |
| | AGGGACT (49.8) (SEQ ID NO:16) | TCGTGCGCATGTAAAACGACGGCCAGT | GGGCTCCTCGAGGACAGGGAC[T/C]G |
| | CACTCACGCAAACGGGTCGAGGACAG | (53.2) (SEQ ID NO:18) | GCCTGTCTGTCCACTGCTGTAACCCC |
| | GGACC (53.6) (SEQ ID NO:17) | | GCGCCGGCATAGGGACCTAAGG |
| | | | (SEQ ID NOs:72 and 85) |
| 06_04 | AACTCTCTCCCAAGAGCGATTCCTTATT | P-GTATATGGATACATGGCTGTCCTGCTG | CACAAATTTGCACATAAAGAATGTCAC |
| | TGATTGCT (49.3) (SEQ ID NO:21) | TTGCATGGCATCTGTAAAACGACGGCCA | GAACAGCCATGTATCCATATAC[A/G]G |
| | CACTCACGCAAACGGGTTCCTTATTTG | GT (54.2) (SEQ ID NO:23) | CAATCAAATAAGGAACTTATGACCTAA |
| | ATTGCC (52.5) (SEQ ID NO:22) | | AGCAAAGGTAAACTTTCTTG (SEQ ID |
| | | | NOs:73 and 86) |
| 07_02 | AACTCTCTCCCAAGAGCGATGACGGCT | P-GAGAGCATATCTAAAAAACAGATGGCT | TGCATGTGACCAAATGTTTGCAGAGTG |
| | CACC (47.4) (SEQ ID NO:26) | TTTAGGAACGCGCATGTAAAACGACGGC | TTTTTTAGATATGCTCTC[G/A]GTGAGC |
| | CACTCACGCAAACGGGATGACGGCTCA | CAGT (52.1) (SEQ ID NO:28) | CGTCATCCGTCAATCCAAGAAGAAGAT |
| | CT (43.2) (SEQ ID NO:27) | | GTTGTTGAACT (SEQ ID NOs:74 |
| | | | and 87) |
| 10_03 | AACTCTCTCCCAAGAGCGATACACGGC | P-GAAAATTATGATCTTTGTTAGGATCAC | GCTTCAATCACATAAACATTCATTGCA |
| | TAATCATT (46.6) (SEQ ID NO:31) | CGTTACCGTCCCGCATGTAAAACGACGG | GGAGTTACACGGCTAATCAT[T/G]GAA |
| | CACTCACGCAAACGGGTACACGGCTAA | CCAGT (51.8) (SEQ ID NO:33) | AATTATGATCTTTGTTAGCTTAAAAGAA |
| | TCATG (47.4) (SEQ ID NO:32) | | AATTCAGTTTAATACAAA (SEQ ID |
| | | | NOs: 75 and 88) |

Multiplex Probe Sets

| | | | |
|---|---|---|---|
| 10_05 | AACTCTCTCCCAAGAGCGATCCAACCA | P-TTCTGCTTCAATAAATCTTCGCAAGAC | TTTGAGTTTTTCCAAAGATTTATTGAAG |
| | ACTTGG (47.9) (SEQ ID NO: 36) | AGGATTTAGGCGCATGTAAAACGACGGC | CAGAA[C/A]CAAGTTGGTTGGATACTT |
| | CACTCACGCAAACGGGATCCAACCAAC | CAGT (52.0) (SEQ ID NO: 38) | GCTGGAAAAAAAAAAGCA (SEQ ID |
| | TTGT (43.5) (SEQ ID NO: 37) | | NOs: 76 and 89) |
| 11_02 | AACTCTCTCCCAAGAGCGACCGCTCTG | P-GACAGGCAGAGCAAAGGTGCTGGCT | TAGATGGGCAGCAGAGGCAACTCCCG |
| | ACCACC (51.6) (SEQ ID NO: 41) | GGATTATGGCGATGTAAAACGACGGCCA | CATCCTTTGCTCTGCGTGTG[G/A]GTG |
| | CACTCACGCAAACGGGCCGCTCTGACC | GT (57.8) (SEQ ID NO: 43) | GTCAGAGCGGTGAGCGAGGTGGGTT |
| | ACT (47.5) (SEQ ID NO: 42) | | GGAGACTCAGCAGGCTCC (SEQ ID |
| | | | NOs: 77 and 90) |
| 11_03 | AACTCTCTCCCAAGAGCGATTAGGTGC | P-TTTATTTTCCACGGATGGAACGATCAC | GATGGAAGTTTGATTCTTCAGATTGTG |
| | TAAACCG (47.2) (SEQ ID NO: 46) | GTGCGCAACGATGTAAAACGACGGCCAGT | ACTCATCCGTGGAAAATAAA[C/T]GGT |
| | CACTCACGCAAACGGGTTAGGTGCTAA | (53.3) (SEQ ID NO: 48) | TTAGCACCTAAATCTGTATATTCCCAT |
| | ACCA (43.5) (SEQ ID NO: 47) | | CAGTGGCTTGGCTGACTCA (SEQ ID |
| | | | NOs: 78 and 91) |
| 12_01 | AACTCTCTCCCAAGAGCGATTGGCAGC | P-TTGCCTGTGATAAGTTGCAAGCACAG | GGCACCTACCGCCAGCTCTTCCACCC |
| | ATCTTCT (49.0) (SEQ ID NO: 51) | CGATGGCTGATTGTAAAACGACGGCCAGT | TGAGCAACTTATCACAGGCAA[A/G]GA |
| | CACTCACGCAAACGGGTTGGCAGCATC | (56.2) (SEQ ID NO: 53) | AGATGCTGCCAATAACTATGCCCGAG |
| | TTCC (52.7) (SEQ ID NO: 52) | | GGCACTACACCATTGGCA (SEQ ID |
| | | | NOs: 79 and 92) |
| 17_02 | AACTCTCTCCCAAGAGCGATGTGCAGG | P-TTTGCTGGATTAGAGGACAGTTCGCA | GAAGTCATGGAAACAGACGGCGGCAC |
| | GAATCAT (48.6) (SEQ ID NO: 56) | AGGCTGGCTGGACATGTAAAACGACGGC | CTTTCCTCTAATCCAGCAAA[A/G]TGAT |
| | CACTCACGCAAACGGGATGTGCAGGG | CAGT (52.4) (SEQ ID NO: 58) | TCCCTGCACACCAGAGACAAGCAGAG |
| | AATCAC (48.8) (SEQ ID NO: 57) | | TAACAGGATCAGTGGGTCTA (SEQ ID |
| | | | NOs: 80 and 93) |
| 18_04 | AACTCTCTCCCAAGAGCGATTAAAAGA | P-CCCCTCCCTTTCTTACAGTTCGCACTC | ATCAAATACTTCATCATAGGCTGAACA |
| | GCAAAGTTT (48.7) (SEQ ID NO: 61) | GCAACTCCGCATGTAAAACGACGGCCAGT | TAATTATTAAAAGAGCAAAGTT[T/A]CC |
| | CACTCACGCAAACGGGATTAAAAGAGC | (53.0) (SEQ ID NO: 63) | CCTCCCTTTCTTACTTTCAAACAAAAC |
| | AAAGTTA (46.9) (SEQ ID NO: 62) | | CAAAAGAGTAGTTTTCATCT (SEQ ID |
| | | | NOs: 81 and 94) |

The primer-specific portion of each probe is shown in italics. The addressable support-specific portion of each second probe is underlined. The pivotal nucleotides fro the two alleles of each biallelic target sequence are in bold and bracketed.

46.5° C for 4.5 minutes for 15 cycles, then incubated at 99° C. for 10 minutes and then at 4° C. The opposing ends of adjacently hybridized target-specific probes were ligated together forming ligation products in the ligation reaction mixture. The skilled artisan will understand that the ligation temperature may be increased or decreased depending on the Tₘ of the first and second probes in the probe sets and that cycle times may also be adjusted accordingly.

C. Purifying the Ligation Product Using Hybridization-Based Pullout

The ligation product was purified as follows. Five I of the ligation reaction mixture, 5 I 1× TE buffer, pH 8.0, and 10 I 2× hybridization buffer (1.8 M tetramethyl ammonium chloride, 0.1 M Tris-HCl, pH 8.0, 0.003 M EDTA, 0.1% Tween 20) were mixed using a micropipette. This mixture was added to a first microtiter plate comprising a nucleotide sequence complementary to one first primer portion of the ligation products and incubated at 41° C. for ten minutes.

The first microtiter plate was placed directly on top of a second microtiter plate comprising a nucleotide sequence complementary to the other first primer portion of the ligation products. The stacked microtiter plates were centrifuged for five minutes at 1480-1600× g in a Beckman Allegra 6KR centrifuge to transfer the unhybridized reaction mixture to the second microtiter plate.

The second microtiter plate was incubated at 41° C. for ten minutes and then placed directly on top of a collection plate. The stacked second microtiter plate and collection plate were centrifuged for five minutes at 1480-1600× g in a Beckman Allegra 6KR centrifuge. The first and second microtiters plates were each washed twice with 50 I of 75% isopropanol, centrifuging for 5 minutes at 1480-1600× g after each wash. The microtiter plates were then washed with 50 I of absolute isopropanol and centrifuged for 5 minutes at 1480-1600× g as before. The microtiter plates were incubated at 37° C. for 15 minutes to dry any residual isopropanol.

The washed first and second microtiter plates were stacked directly on top of collection plates and 30 I of freshly prepared ammonium hydroxide solution (70% ammonium hydroxide v/v in sterile filtered, deionized water) was added to each well. The stacked plates were centrifuged for 5 minutes at 1480-1600× g as before. The eluates, comprising the purified ligation products, were combined in a 0.5 ml microcentrifuge tube and air-dried under vacuum.

D. Amplification of the Purified Ligation Product Using PCR

The purified ligation product was amplified by PCR as follows. The air-dried purified ligation products were rehydrated with 2 I water. The amplification reaction mixture was prepared by combining 2 μl of the rehydrated purified ligation product with 28 I PCR buffer (9 I sterile filtered, deionized water, 18 μl True Allele PCR pre mix (P/N 403061, PE Biosystems, Foster City, Calif.), and 1 μl universal primer mix (30 μM of each primer in 1× TE buffer)).

One first primer (5′-AACTCTCTCCCAAGAGCGA; Tₘ 53.7° C.) (SEQ ID NO: 66) was 5′-end labeled with Ben Joda (3-(4-carboxybenzyl)-13-(3-sulfopropyl)-1,2,3,13,14,15-hexahydro-1,1,15,15 tetramethyl-dibenzo[g,g′]pyrano[2,3-e:6,5-e′]diindol-16-ium, inner salt, carboxy NHS ester). The other first primer (5′-CACTCACGCAAACGGG; Tₘ 53.7° C.) (SEQ ID NO: 67) was 5′-end labeled with VIC (2′-phenyl-7′-chloro-6-carboxy-4,7-dichlorofluorescein) according to the manufacturer's protocols (PE Biosystems, Foster City, Calif.). The second primer (5′-ACTGGCCGTCGTTTTACA; Tₘ 53.7° C.) (SEQ ID NO: 68) was not labeled.

The amplification reaction mixture was then subjected to cycles of amplification in a thermal cycler as follows. The tubes were heated to 95° C. for 12 minutes, then ten cycles of (94° C. for 15 seconds, 60° C. for 15 seconds, and 72° C. for 30 seconds), followed by twenty-five cycles of (89° C. for 15 seconds, 53° C. for 15 seconds, and 72° C. for 30 seconds), and then 45 minutes at 60° C. The amplification reaction mixture, containing double-stranded amplification product, was then cooled to 4° C.

Unincorporated PCR primers were removed from the reaction mixture as follows. To each 30 μl amplification reaction mixture, 0.34 μl of glycogen (10 mg/ml), 3.09 μl 3 M sodium acetate buffer, pH 5, and 20.6 μl absolute isopropanol were added. The tubes were mixed by vortexing and incubated at room temperature for ten minutes followed by centrifugation at 14,000 rpm for 10-15 minutes in a Beckman Model 18 microfuge.

Supernatants were removed from the labeled amplification product pellets. Each pellet was washed with 50 μl of 70% ethanol (in water) with vortexing. The washed amplification products were centrifuged at 14,000 rpm for 5 minutes in a Beckman Model 18 microfuge and the supernatant was removed. The pellets were washed again using 50 μl anhydrous ethanol, vortexed, and centrifuged at 14,000 rpm for 5 minutes in a Beckman Model 18 microfuge. The pellets were air-dried. These air-dried pellets may be stored at 4° C. prior to hybridization.

E. Support Hybridization Using a DNA Microarray

The air-dried amplification product pellets were resuspended in 10 μl 1× TE buffer and combined with 30 μl of hybridization buffer (0.1 M tetramethyl ammonium chloride, 0.5 M MES-sodium salt, pH 6.7, 1% Triton X-100, 10 mg/ml sheared herring sperm DNA (Sigma Chemical Co., St. Louis, Mo.), and 20 mg/ml bovine serum albumin). The tubes were incubated at 94° C. for 10 minutes to generate single-stranded amplification product in the reaction mixture, and then quenched at 4° C.

Fifteen microliters of the reaction mixture containing single-stranded amplification product were added to chambers of a DNA microarray (Hyseq, Sunnyvale, Calif.). The array was placed in the hybridization chamber and incubated at 60° C. for 3 hours to allow the addressable support-specific portions of the single-stranded amplification product to hybridize to the support-bound capture oligonucleotides (MAXI 14, Hybaid, Ashford, Middlesex, UK). The array was washed with wash buffer (300 mM Bicine, pH 8.0, 10 mM MgCl₂, and 0.1% SDS), rinsed with 6× SSPE (0.9 M NaCl, 0.06 M NaH₂PO₄, pH 7.4, 0.02 M EDTA) to remove the wash buffer, and air-dried.

F. Detection of Hybridized Amplification Product

The dried array was placed in an array scanner (GenePix 4000A, Axon Instruments, Foster City, Calif.), scanned at 532 nm and 635 nm, and the presence of labeled amplification products hybridized at specific locations on the array was detected. Detection of a labeled amplification product hybridized to a particular capture probe at a specific array location (address) indicates that the corresponding target sequence is present in the sample. In certain embodiments, the amplification products are distinguished not by the mere presence of a detectable label at an array address, but by the particular label or combination of labels that are detected.

The skilled artisan will appreciate that the complement of the disclosed probe, target, and primer sequences, or combinations thereof, may be employed in the methods of invention. For example, without limitation, a genomic DNA sample comprises both the target sequence and its complement. Thus when a genomic sample is denatured, both the target sequence and its complement are present in the sample as single stranded sequences. The probes described herein will specifically hybridize to the appropriate sequence, either the target or its complement.

Although the invention has been described with reference to various applications, methods, and compositions, it will be appreciated that various changes and modifications may be made without departing from the invention.

1-114. (canceled)

1. A method for detecting one or more target sequences in a sample comprising:
combining the sample with a probe set for each target sequence, the probe set comprising (a) at least one first probe, comprising a target-specific portion and a 5′ primer-specific portion, and (b) at least one second probe, comprising a target-specific portion and a 3′ primer-specific portion, wherein the probes in each set are suitable for ligation together when hybridized adjacent to one another on a complementary target sequence, and wherein at least one probe in each probe set further comprises an addressable support-specific portion located between the primer-specific portion and the target-specific portion; to form a ligation reaction mixture;
subjecting the ligation reaction mixture to at least one cycle of ligation, wherein adjacently hybridizing complementary probes are ligated to one another to form a ligation product comprising the 5′ primer-specific portion, the target-specific portions, at least one addressable support-specific portion, and the 3′ primer-specific portion;
combining the ligation reaction mixture with: (a) at least one primer set, the primer set comprising (i) at least one primer comprising the sequence of the 5′ primer-specific portion of the ligation product, and (ii) at least one second primer comprising a sequence complementary to the 3′ primer-specific portion of the ligation product, and (b) a polymerase, to form an amplification reaction mixture;
subjecting the amplification reaction mixture to at least one cycle of amplification to generate one or more amplification products;
combining the one or more amplification products with at least two different sequence-specific mobility-modifiers, wherein each different mobility-modifier is capable of sequence-specific binding to a different addressable support-specific portion and comprises (a) a tag complement for specifically binding the addressable support-specific portion of one of the one or more amplification products, and (b) a tail which imparts to each mobility modifier a mobility that is distinctive relative to the mobilities of one or more other of said at least two different mobility-modifiers in a mobility-dependent analysis technique;
removing mobility-modifiers that are not sequence-specifically bound to the one or more amplification products from mobility-modifiers that are sequence-specifically bound to the one or more amplification products;
releasing the sequence-specifically bound mobility-modifiers from the one or more amplification products;
subjecting the released mobility-modifiers to a mobility-dependent analysis technique; and
detecting the one or more target sequences in the sample by detecting distinctive positions of the mobility-modifiers.

2. The method of claim 1, wherein at least one sequence-specific mobility modifier comprises a label.

3. The method of claim 1, wherein the mobility-dependent analysis technique is electrophoresis.
